(19) United States
(12) Patent Application Publication
(10) Pub. No.: US 2003/0224435 A1
(43) Pub. Date: Dec. 4, 2003

(54) Detection of abused substances and their metabolites using nucleic acid sensor molecules

(76) Inventor: Scott Seiwert, Pacifica, CA (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 10/439,715

(22) Filed: May 16, 2003

Related U.S. Application Data
(60) Provisional application No. 60/381,006, filed on May 16, 2002.

(51) Int. Cl. C12Q 1/68

(52) U.S. Cl. 435/6

(57) ABSTRACT

Nucleic acid sensor molecules (allozymes, allosteric ribozymes, allosteric DNAzymes), aptamers and methods are provided for the detection and quantitation of small molecules, including drugs, drug analogs, and drug metabolites, for example recreational drugs, mood-altering drugs, and performance enhancing drugs such as 4-MTA (4-methylthioamphetamine), Alpha-ethyltryptamine, Amphetamine, Amyl nitrite, Benzocaine, Cocaine, Dimethyltryptamine, Ecstasy (MDA, MDMA, MDEA), Ephedrine, Erythropoietine (Epogen), Fentanyl, Gamma Hydroxybutyrate (GHB), GBL (Gamma butyrolactone), GHB (Gamma Hydroxybutyrate), Hashish, Heroin, Isobutyl nitrite, Ketamine, Lidocaine, LSD (Lysergic acid diethylamide), Mannitol, Marijuana (THC), Mescaline, Methadone, Methamphetamine, Methaqualone, Methcathinone, Methylphenidate (ritalin), Morphine, Nexus (2CB), Nicotine, Opium, Oxycodone, OxyContin, PCP (phencyclidine), Peyote, Phenobarbital, Procaine, Psilocybin, Psilocybin/psilocin, Pseudoephedrine, Rohypnol, Scopolamine, Steroids, Strychnine, and Talwin. Also provided are kits for detection. The nucleic acid sensor molecules, methods and kits provided herein can be used in diagnositic applications for detecting drugs, analogs, and metabolites thereof.

This patent application claims the benefit of U.S. Ser. No. 60/381,006, filed May 16, 2002. This application is hereby incorporated by reference herein in its entirety including the drawings.

### FIELD OF THE INVENTION

This invention relates generally to the field of drug and drug metabolite detection in biological samples. More specifically, it provides a system for detecting or confirming the presence of a particular drug analyte in a sample that potentially contains interfering substances. This invention specifically relates to novel molecular sensors that utilize enzymatic nucleic acid constructs whose activity can be modulated by the presence or absence of signaling agents that include compounds and substances of abuse, such as recreational drugs, mood altering drugs, performance enhancing drugs, analgesics, and metabolites thereof. The present invention further relates to the use of the enzymatic nucleic acid constructs as molecular sensors capable of modulating the activity, function, or physical properties of other molecules useful in detecting compounds and substances of abuse and metabolites thereof. The invention also relates to the use of the enzymatic nucleic acid constructs as diagnostic reagents, useful in identifying such signaling agents in a variety of applications, for example, in screening biological samples or fluids for compounds and substances of abuse and metabolites thereof.

### BACKGROUND OF THE INVENTION

The ability to perform rapid screening tests in diagnostic analysis of biological samples has been considerably facilitated by the evolving art of immunoassay. Antibodies can be raised that have exquisite specificity and sensitivity for small molecules of diagnostic interest, such as drugs and drug metabolites. In combination with other reagents that have a separating or labeling function, specific antibodies can be used as part of a rapid screening test for the presence of the small molecule in a clinical sample. Similarly, nucleic acid technology can be applied to develop polynucleotide based detection systems comprising nucleic acid molecules with high affinity for a particular small molecule target. Furthermore, the functionality of enzymatic nucleic acid molecules can be coupled with these recognition properties in the design of nucleic acid sensor molecules having both recognition and signal generating capability.

Of increasing interest are diagnostic applications of nucleic acid molecules, such as aptamers and allosteric ribozymes. These molecules offer many advantages over traditional protein antibodies for use as diagnostic agents. For example, nucleic acids can be designed, developed, and manufactured more rapidly than protein antibodies. In addition, nucleic acid molecules can be synthesized with less expense than protein molecules. Because nucleic acid sensor molecules can be evolved to recognize a target in vitro, the recognition properties of the nucleic acid are readily modulated to recognize a single molecule or alternately, members of a class of molecules. Nucleic acid molecules can also be chemically modified to modulate their activity. The detection of small molecules, including drugs and drug metabolites, therefore represents an ideal application for nucleic acid molecules having ligand recognition properties, since these molecules offer exceptional specificity and can be designed to detect subtle variations in the structure of a target analyte or class of analytes.

Small molecules that can be assayed in this manner include hormones, natural metabolites, prescription drugs, non-prescription drugs, and illicit drugs. In particular, nucleic acid molecules can be used to detect substances of abuse, including the inappropriate voluntary use of recreational drugs and performance enhancing drugs. Substances of abuse include canabinoids; tranquilizers, such as barbiturates; stimulants, such as amphetamines; opiates, such as heroin, morphine, codiene, and oxycodine; analgesics, such as oxycontin; hallucinogenic alkaloids, such as cocaine, ecstasy (MDMA and equivalents), phencyclidine (PCP), and lysergic acid diethylamide (LSD); and performance enhancing drugs, such as anabolic steroids and epogen.

Cubicciotti, U.S. Pat. No. 6,287,765, describes certain methods for detecting and identifying single molecules.

Stojanovic et al., 2000, J. Am. Chem. Soc., 122, 46, describes certain cocaine sensing nucleic acid aptamers.

George et al., U.S. Pat. Nos. 5,834,186 and 5,741,679, describe regulatable RNA molecules whose activity is altered in the presence of a ligand.

Shih et al., U.S. Pat. No. 5,589,332, describe a method for the use of ribozymes to detect macromolecules such as proteins and nucleic acid.

Nathan et al., U.S. Pat. No. 5,871,914, describe a method for detecting the presence of an assayed nucleic acid based on a two component ribozyme system containing a detection ensemble and an RNA amplification ensemble.

Nathan and Ellington, International PCT publication No. WO 00/24931, describe the detection of an analyte by a catalytic nucleic acid sequence which converts a nucleic acid substrate to a catalytic nucleic acid product in the presence of the analyte. The catalytic nucleic acid product is then amplified, by PCR.

Sullenger et al., International PCT publication No. WO 99/29842, describe nucleic acid mediated RNA tagging and RNA revision.

Nathan et al., International PCT Publication No. WO 98/08974, describes specific cofactor-dependent ribozyme constructs.

Usman et al., International PCT Publication No. WO 01/66721, describes nucleic acid sensor molecules.

### SUMMARY OF THE INVENTION

This invention relates to novel molecular sensors that utilize enzymatic nucleic acid constructs whose activity can be modulated by the presence or absence of signaling agents that include compounds and substances of abuse, such as recreational drugs, mood altering drugs, analgesics, performance enhancing drugs and metabolites thereof. The present invention further relates to the use of the enzymatic nucleic acid constructs as molecular sensors capable of modulating the activity, function, or physical properties of other molecules useful in detecting compounds and substances of abuse and metabolites thereof. The invention also relates to the use of the enzymatic nucleic acid constructs as diagnostic reagents, useful in identifying such signaling agents in a variety of applications, for example, in screening biological samples or fluids for compounds and substances of abuse and metabolites thereof.

In one embodiment, the invention features a nucleic acid sensor molecule that is used to assay the presence of a drug or drug metabolite in a system or sample, such as a biological system or sample. Non-limiting examples of drug compounds contemplated by the instant invention for detection with nucleic acid sensor moleules are shown in Table 1. The invention further contemplates analogs, isomers, and metabolites of the compounds generally referred in Table 1. Thecompounds and analogs, isomers, and metabolites thereof are generally referred to herein as target signalling molecules. Nucleic acid sensor molecules of the invention are generally described in George et al., U.S. Pat. Nos. 5,834,186 and 5,741,679, Shih et al., U.S. Pat. No. 5,589,332, and Usman et al., U.S. Ser. No. 09/800,594, filed Mar. 6, 2001 and Usman et al., U.S. Ser. No. 09/877,526, filed Jun. 8, 2001 both incorporated by reference herein in their entirety, including the drawings.

In another embodiment, the invention features a nucleic acid sensor molecule having specificity for a single compound comprising a drug or drug metabolite. In yet another embodiment, the invention features a nucleic acid sensor molecule having specificity for a class of compounds comprising a drug, class of drugs, or metabolites thereof.

In one embodiment, the invention features a nucleic acid aptamer molecule that is used to assay for the presence of a drug or drug metabolite in a system or sample, such as a biological system or sample. Non-limiting examples of drug compounds contemplated by the instant invention for detection with nucleic acid aptamer moleules are shown in Table 1, including analogs, isomers, and metabolites thereof.

In another embodiment, the invention features a nucleic acid aptamer molecule having specificity for a single compound comprising a drug or drug metabolite. In yet another embodiment, the invention features a nucleic acid aptamer molecule having specificity for a class of compounds comprising a drug, class of drugs, or metabolites thereof.

In one embodiment, the nucleic acid molecule of the invention is a linear nucleic acid molecule. In another embodiment, the nucleic acid molecule of the invention is a linear nucleic acid molecule that can optionally form a hairpin, loop, stem-loop, or other secondary structure. In yet another embodiment, the nucleic acid molecule of the invention is a circular nucleic acid molecule.

In another embodiment, the nucleic acid molecule of the invention is a single stranded oligonucleotide. In another embodiment, the nucleic acid molecule of the invention is a double-stranded oligonucleotide.

In one embodiment, the nucleic acid sensor molecule of the invention comprises an oligonucleotide having between about 3 and about 500 nucleotides. In another embodiment, the nucleic acid sensor molecule of the invention comprises an oligonucleotide having between about 4 and about 100 nucleotides. In another embodiment, the nucleic acid sensor molecule of the invention comprises an oligonucleotide having between about 5 and about 50 nucleotides.

In another embodiment, the nucleic acid aptamer of the invention comprises an oligonucleotide having between about 3 and about 100 nucleotides. In another embodiment, the nucleic acid aptamer of the invention comprises an oligonucleotide having between about 4 and about 50 nucleotides. In another embodiment, the nucleic acid aptamer of the invention comprises an oligonucleotide having between about 5 and about 30 nucleotides.

In one embodiment, the invention features a method for identifying nucleic acid aptamers of the invention having binding affinity for a target drug or drug metabolite, comprising: (a) generating a randomized pool of oligonucleotides; (b) combining the oligonucleotides from (a) with the target drug or drug metabolite under conditions suitable to allow at least one oligonucleotide in the pool to bind to the target drug or drug metabolite; (c) partitioning oligonucleotide sequences (ligands) that bind to the target drug or drug metabolite and unbound oligonucleotide sequences; (d) amplifying the oligonucleotide sequences isolated from (c) that bind to the target drug or drug metabolite; (e) combining the oligonucleotides from (d) with the target drug or drug metabolite under conditions suitable to allow at least one oligonucleotide to bind to the target drug or drug metabolite; and (f) repeating steps (c), (d), and (e) under conditions suitable for isolating one or more nucleic acid molecules having binding affinity to the target drug or drug metabolite. In another embodiment, step (d) is optionally carried out under conditions suitable for introducing some degree of mutation into the sequences in step (d).

In one embodiment, the invention features a method for generating nucleic acid sensor molecules capable of detecting the presence of a target drug or drug metabolite in a system, comprising: (a) coupling a nucleic acid aptamer of the invention to an enzymatic nucleic acid molecule via a randomized nucleic acid sequence; (b) combining the oligonucleotides from (a) with the target drug or drug metabolite in vitro under conditions suitable to allow target binding mediated catalysis of the enzymatic nucleic acid molecule; (c) isolating oligonucleotide sequences from (b) that possess catalytic activity by removing inactive oligonucleotide sequences; (d) amplifying the oligonucleotide sequences isolated from (c); and (e) repeating steps (c) and (d) under conditions suitable for isolating one or more nucleic acid sensor molecules having catalytic activity in the presence of the target drug or drug metabolite. In another embodiment, step (d) is optionally carried out under conditions suitable for introducing some degree of mutation into the sequences. In another embodiment, the random region of (a) comprises a single stranded sequence. In yet another embodiment, the randomized nucleic acid sequence of (a) comprises a double stranded stem or stem loop structure.

In another embodiment, the invention features a method for generating nucleic acid sensor molecules capable of detecting the presence of a target drug or drug metabolite in a system, comprising: (a) generating a pool of nucleic acid sequences having an enzymatic nucleic acid domain and a target binding domain comprising one or more random regions of nucleotides; (b) combining the oligonucleotides from (a) with the target in vitro under conditions suitable to allow target binding mediated catalysis of the enzymatic nucleic acid molecule; (c) isolating oligonucleotide sequences from (b) that possess catalytic activity by removing inactive oligonucleotide sequences; (d) amplifying the oligonucleotide sequences isolated from (c); and (e) repeating steps (c) and (d) under conditions suitable for isolating one or more nucleic acid sensor molecules having catalytic activity in the presence of the target drug or drug metabolite. In another embodiment, step (d) is optionally carried out under conditions suitable for introducing some degree of mutation into the sequences. In another embodiment, a random region of (a) comprises a single stranded sequence. In yet another embodiment, a random region of (a) comprises a double stranded stem or stem loop structure.

In another embodiment, the invention features a method for generating nucleic acid sensor molecules capable of detecting the presence of a target drug or drug metabolite in a system, comprising: (a) generating a pool of random nucleic acid sequences; (b) combining the oligonucleotides from (a) with the target in vitro under conditions suitable to allow target mediated catalysis of the enzymatic nucleic acid molecule; (c) isolating oligonucleotide sequences from (b) that possess catalytic activity by removing inactive oligonucleotide sequences; (d) amplifying the oligonucleotide sequences isolated from (c); and (e) repeating steps (c) and (d) under conditions suitable for isolating one or more nucleic acid sensor molecules having catalytic activity in the presence of the target drug or drug metabolite. In another embodiment, step (d) is optionally carried out under conditions suitable for introducing some degree of mutation into the sequences.

In another embodiment, the methods of selecting nucleic acid sensor molecules of the invention utilize cis cleavage of a reporter molecule that comprises a fixed nucleotide sequence for purposes of selection. In yet another embodiment, the methods of selecting nucleic acid sensor molecules of the invention utilize trans cleavage of a reporter molecule having a fixed sequence for purposes of selection.

In one embodiment, methods of the invention are applied to generate nucleic acid sensor molecules that are inactive in the presence of the target drug or drug metabolite, for example, by selecting nucleic acid sensor molecules whose activity is inhibited in the presence of the target drug or drug metabolite.

In the described methods, the random pool of oligonucleotides in the above methods can comprise DNA and/or RNA, with or without chemically modified nucleotides. When chemically modified nucleotides are used in the method, such modifications can be chosen such that a non-discriminatory polymerase will incorporate the chemically modified nucleotide into the oligonucleotide sequence when generated or amplified. Non-limiting examples of chemically modified nucleoside triphosphates (NTPs) that can be used in the method of the invention include 2′-deoxy-2′-fluoro, 2′-deoxy-2′-amino, 2′-O-alkyl, and 2′-O-methyl NTPs as well as various base modified NTPs, such as C5-modified pyrimidines, 2,6-diaminopurine, and inosine. The oligonucleotides used in the method can be of fixed or variable length.

In one embodiment, the target drug or drug metabolite used in the methods above can be a drug referred to in Table 1, or an analog or metabolite thereof. Such analogs and metabolites can include, for example, substitutions, deletions, or additions of functional groups, atoms, or ions.

In another embodiment, the method for identifying nucleic acid acids of the invention comprises attaching the target drug or drug metabolite to a solid matrix, such as beads, microtiter plate wells, membranes, chip surfaces, or other solid matrices known in the art. In such a system, the target drug or drug metabolite can be attached to the solid matrix either covalently or non-covalently. In yet another embodiment, the oligonucleotide or nucleic acid used in a method of the invention can be labeled, either directly or non-directly, for example, with a radioactive label, absorption label such as biotin, or a fluorescent label such as fluorescein or rhodamine.

In one embodiment, the invention features a method for detecting the presence of a drug in a sample comprising: (a) contacting the sample with an enzymatic nucleic acid molecule of the invention, and (b) assaying for the presence of the drug under conditions suitable for detecting the presence of the drug in the sample. Non-limiting examples of samples that are used with the method of the invention include biological samples derived from a subject such as blood, serum, urine, saliva, sputum, hair, cutaneous tissues, and adipose tissue. Such samples can be subjected to various treatment steps further contemplated by the methods herein, including partial purification, filtration, nuetralization, digestion, dilution, concentration, chemical treatment, etc.

In one embodiment, the biological sample is derived from a mammalian subject. In one embodiment, the biological sample is derived from a human subject.

Detecting and/or quantitating the presence of drug in the above inventive method can be accomplished using a variety of methods, including detecting an increase or decrease in fluorescence, an increase or decrease in enzymatic activity, an increase or decrease in the production of a precipitate, an increase or decrease in chemoluminescence, an increase or decrease in chemiluninescence, or likewise a change in UV absorbance, phosphorescence, pH, optical rotation, isomerization, polymerization, temperature, mass, capacitance, resistance, emission of radiation, or colorimetric change.

In another embodiment, the invention features a kit comprising a nucleic acid sensor molecule of the invention. The kit of the invention can further include any additional reagents, reporter molecules, buffers, excipients, containers and/or devices as required to practice a method of the invention.

Detecting and/or quantitating the presence of drug in the above inventive method can be accomplished using a reporter molecule. The reporter molecule can be attached to the inventive enzymatic nucleic acid molecule or can be free in the sample. In one embodiment, the reporter molecule of the instant invention comprises a detectable label selected from the group consisting of chromogenic substrate, fluorescent labels, chemiluminescent labels, and radioactive labels and enzymes. Suitable enzymes include, for example, luciferase, horseradish peroxidase, and alkaline phosphatase.

In another embodiment, the reporter molecule of the instant invention is immobilized on a solid support. Suitable solid supports include silicon-based chips, silicon-based beads, controlled pore glass, polystyrene, cross-linked polystyrene, nitrocellulose, biotin, plastics, metals and polyethylene films.

In one embodiment, the invention features an array of nucleic acid sensor molecules comprising a predetermined number of nucleic acid sensor molecules of the invention. In one embodiment, a nucleic acid sensor molecule of the instant invention is attached to a solid surface. Preferably, the surface of the instant invention comprises silicon-based chips, silicon-based beads, controlled pore glass, polystyrene, cross-linked polystyrene, nitrocellulose, biotin, plastics, metals and/or polyethylene films.

In one embodiment, the range of detection for a method of the invention is from 1 to 4000 ng/ml of the target compound in urine, saliva, or blood. In another embodiment, the range of detection for a method of the invention is from 100 to 5,000 ug/l of the target compound in urine, saliva, or blood.

In one embodiment, any of the inventive methods is carried out more than once.

### BRIEF DESCRIPTION OF THE DRAWINGS

FIG. 1 shows a non-limiting diagrammatic example of a method for generating nucleic acid sensor molecules of the invention from a completely random pool of nucleic acid sequences (but having fixed binding arm sequences for interaction with a reporter molecule). The method comprises: (1) generating a random pool of nucleic acid sequences, (2) discarding any active sequences that have catalytic activity in the absense of target, (3) adding target to the pool, (4) discarding molecules that are inactive in the presence of the target, (5) amplification to enrich nucleic acid sensor sequences, and (6) repeating the process of (1-5) to increase nucleic acid sensor sensitivity and catalytic activity.

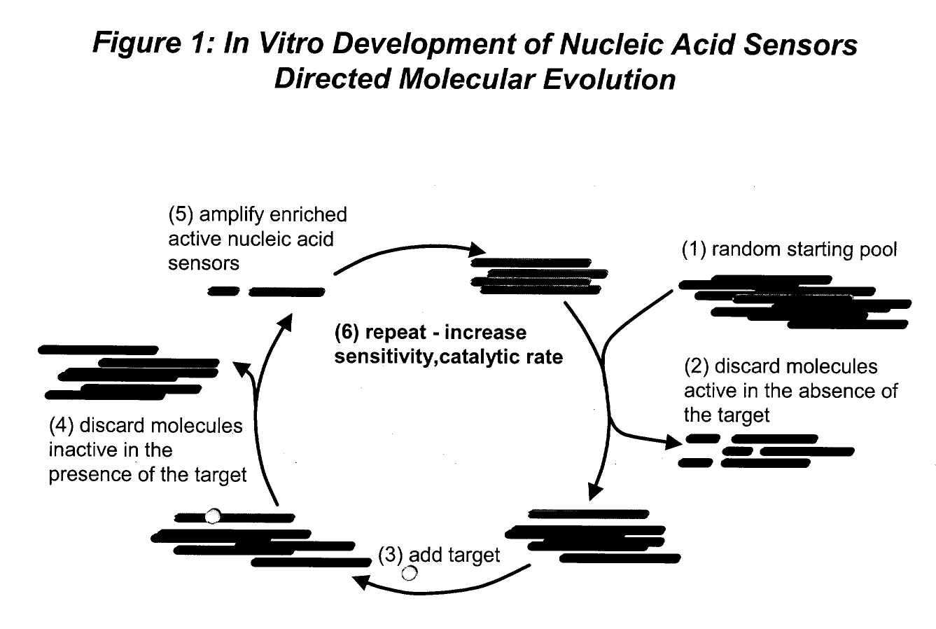

FIG. 2 shows a graph depicting the pharmacokinetics of 3,4-methylenedioxymethamphetamine (MDMA) as described by De La Torre et al., 2000, J. Clinical Pharmacology, 49, 104-109. This example shows that levels of MDMA (ecstasy) in saliva and plasma are well suited for detection using nucleic acid sensor molecules.

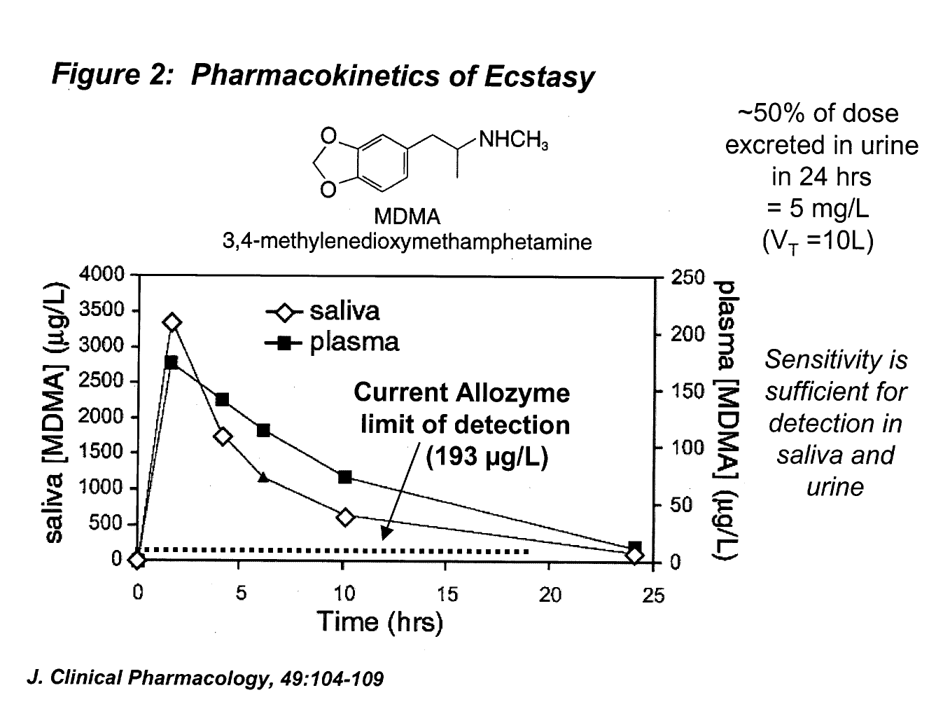

FIG. 3 shows a non-limiting example of a fluorescence resonance energy transfer (FRET) solution phase assay format. In the absense of a target molecule, the nucleic acid sensor molecule is inactive. In the presense of a target molecule, the nucleic acid sensor molecule is active, and cleaves a substrate reporter molecule comprising a nucleic acid sequence having a fluorophore (D) and quencher moiety (A). Once the reporter molecule is cleaved, the distance between the fluorophore and quencher moiety is increased, resulting is fluorescence and signal generation. Different fluorophores (Cy3, Rox, Cy5, and Cy7) have different wavelengths for detection, thereby allowing multiplexed assays for different drug targets within the same assay.

FIG. 4 shows a non-limiting example of a colorimetric solution phase assay format. In the absense of a target molecule, the nucleic acid sensor molecule is inactive. In the presense of a target molecule, the nucleic acid sensor molecule is active, and cleaves a substrate reporter molecule comprising a nucleic acid sequence having a terminal colorimetric group, such as a para-nitrophenyl group. Once the reporter molecule is cleaved, the colorimetric group is released (such as p-nitrophenol), generating a detectable color.

FIG. 5 shows chemical structures of common forms of the drug “ecstasy”, including 3,4-methylenedioxyamphetamine (MDA), 3,4-methylenedioxymethamphetamine (MDMA), and 3,4-methylenedioxy-N-ethyl-amphetamine (MDEA) as compared to related drug compounds ampethamine (AMP) and methampehtamine (METAMP) and the common metabolite of ecstasy, 4-hydroxy-3-methoxy-methamphetamine (HMMA). Nucleic acid sensor molecules and aptamers of the invention can be designed to recognize the common class of esctasy drugs or individual members of the ecstasy family as distinguished from amphetamine, methamphetamine, and/or 4-hydroxy-3-methoxy-methamphetamine.

FIG. 6 shows a non-limiting diagrammatic example of a method for generating nucleic acid sensor molecules of the invention using a partially defined sequence comprising a known enzymatic nucleic acid molecule coupled with a randomized sensor region represented by Ns in the figure. The method comprises: (1) generating a pool of nucleic acid sequences comprising a fixed domain and a random domain, (2) discarding any active sequences that have catalytic activity in the absense of target, (3) adding target to the pool, (4) discarding molecules that are inactive in the presence of the target, (5) amplification to enrich nucleic acid sensor sequences, and (6) repeating the process of (1-5) to increase nucleic acid sensor sensitivity and catalytic activity.

FIG. 7 shows a non-limiting diagrammatic example of a method for generating nucleic acid sensor molecules of the invention using a defined aptamer sequence having specificity for the target molecule coupled to a known enzymatic nucleic acid molecule via a randomized stem sequence represented by Ns in the figure. The method comprises: (1) generating a pool of nucleic acid sequences comprising two fixeded domains (aptamer sensor domain and enzymatic nucleic acid domain) and a random domain (connecting sequence), (2) discarding any active sequences that have catalytic activity in the absense of target, (3) adding target to the pool, (4) discarding molecules that are inactive in the presence of the target, (5) amplification to enrich nucleic acid sensor sequences, and (6) repeating the process of (1-5) to increase nucleic acid sensor sensitivity and catalytic activity.

### DETAILED DESCRIPTION OF THE INVENTION

The present invention features compounds, compositions, methods, and kits for the detection of specific target signalling agents, such as drugs, (exemplary drugs are shown in Table 1, and include drug analogs and drug metabolites thereof) in a system using nucleic acid sensor molecules and nucleic acid aptamers.

In one embodiment, the present invention features a nucleic acid sensor molecule comprising an enzymatic nucleic acid component and one or more sensor components wherein, in response to an interaction with a target signaling agent, the enzymatic nucleic acid component catalyzes a chemical reaction in which the activity or physical properties of a reporter molecule is modulated. Preferably, the chemical reaction in which the activity or physical properties of a reporter molecule is modulated results in a detectable response.

In one embodiment, the present invention features a nucleic acid sensor molecule comprising an enzymatic nucleic acid component and one or more sensor components wherein, in response to an interaction of a target signaling agent with the nucleic acid sensor molecule, the enzymatic nucleic acid component catalyses a chemical reaction involving covalent attachment of at least a portion of a reporter molecule.

The chemical reaction in which a reporter molecule is covalently attached to the nucleic acid sensor molecule can be, for example, a ligation, transesterification, phosphorylation, carbon-carbon bond formation, amide bond formation, peptide bond formation, and disulfide bond formation.

In another embodiment, the present invention features a nucleic acid sensor molecule comprising an enzymatic nucleic acid component and one or more sensor components wherein, in response to an interaction of a target signaling molecule with the nucleic acid sensor molecule, the enzymatic nucleic acid component carries out a chemical reaction that modulates the activity or properties of the reporter molecule. The chemical reaction in which the activity of a reporter molecule is modulated can be, for example, a phosphorylation, dephosphorylation, isomerization, polymerization, amplification, helicase activity, transesterification, ligation, hydration, hydrolysis, alkylation, dealkylation, halogenation, dehalogenation, esterification, desterification, hydrogenation, dehydrogenation, saponification, desaponification, amination, deamination, acylation, deacylation, glycosylation, deglycosylation, silation, desilation, hydroboration, epoxidation, peroxidation, carboxylation, decarboxylation, substitution, elimination, oxidation, and reduction reaction, or any combination of these reactions and like reactions.

In one embodiment, the invention features a nucleic acid sensor molecule comprising an enzymatic nucleic acid component and one or more sensor components wherein, in response to an interaction of a target signaling molecule with the nucleic acid sensor molecule, the enzymatic nucleic acid component can carry out a chemical reaction involving isomerization of at least a portion of a reporter molecule.

In another embodiment, the invention features a nucleic acid sensor molecule comprising an enzymatic nucleic acid component and one or more sensor components wherein, in response to an interaction of a target signaling molecule with the nucleic acid sensor molecule, the enzymatic component catalyses a chemical reaction on a non-oligonucleotide-based portion of a reporter molecule selected from the group consisting of phosphorylation and dephosphorylation reactions.

Nucleic acid sensor molecules of the invention can have a detection signal, such as from a reporter molecule. Examples of reporter molecules include nucleic acid molecules comprising various tags, probes, beacons, fluorophores, chemophores, ionophores, radio-isotopes, photophores, peptides, proteins, enzymes, antibodies, nucleic acids, and enzymatic nucleic acids or a combination thereof. The reporter molecule may optionally be covalently linked to a portion of the nucleic acid sensor molecule.

In another embodiment, the reporter molecule of the instant invention can be a molecular beacon, small molecule, fluorophore, chemophore, ionophore, radio-isotope, photophore, peptide, protein, enzyme, antibody, nucleic acid, and enzymatic nucleic acid or a combination thereof (see, for example, Singh et al., 2000, Biotech., 29, 344; Lizardi et al, U.S. Pat. Nos. 5,652,107 and 5,118,801).

Using such reporter molecules and others known in the art, the detectable response of the instant invention can be monitored by, for example, a change in fluorescence, color change, UV absorbance, phosphorescence, pH, optical rotation, isomerization, polymerization, temperature, mass, capacitance, resistance, emission of radiation and the like.

Detection of the target signaling event via the chemical reaction or the change in activity or physical properties of the reporter molecule can be assayed by methods known in the art. Amplification of the target signaling event via the chemical reaction or the change in activity or physical properties of the reporter molecule can be accomplished by methods known in the art, for example, modulating polymerase activity. Modulation of polymerase activity can increase polymerization in a chemical reaction, for example, a polymerase chain reaction (PCR) system, resulting in amplification of a target signaling molecule or reporter molecule.

In one embodiment, a linker region can join the nucleic acid sensor molecule to a reporter molecule, for example, via ligation activity of an enzymatic nucleic acid component of the nucleic acid sensor molecule in response to a target signaling agent's interaction with a sensor component of the nucleic acid sensor molecule.

In one embodiment, the invention features a nucleic acid sensor molecule having a reporter molecule, wherein said reporter molecule comprises the formula:

R₁—L—R₂

wherein R1 is selected from the group consisting of alkyl, alkoxy, hydrogen, hydroxy, sulfhydryl, ester, anhydride, acid halide, amide, nitrile, phosphate, phosphonate, nucleoside, nucleotide, oligonucleotide; R2 is selected from the group consisting of molecular beacons, small molecules, fluorophores, chemophores, ionophores, radio-isotopes, photophores, peptides, proteins, enzymes, antibodies, nucleic acids, and enzymatic nucleic acids; L represents a linker which can be present or absent, and “-” represents a chemical bond

In another embodiment, the invention features a nucleic acid sensor molecule having a reporter molecule, wherein said reporter molecule comprises the formula:

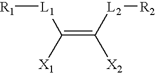

wherein R1 and R2 each represent compounds, which can be the same or different, that generate a detectable signal or quench a detectable signal when an isomerization reaction is catalyzed, selected from the group consisting of molecular beacons, small molecules, fluorophores, chemophores, ionophores, radio-isotopes, photophores, peptides, proteins, enzymes, antibodies, nucleic acids, and enzymatic nucleic acids; L1 and L2 each represent a linker which can be the same or different and which can be present or absent; X1 and X2 each represent an atom, compound, or molecule that can be the same or different, and “-” represents a chemical bond. In another preferred embodiment, the invention features a nucleic acid sensor molecule having a reporter molecule, wherein said reporter molecule comprises the formula:

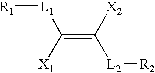

wherein R1 and R2 each represent compounds, which can be the same or different, that generate a detectable signal or quench a detectable signal when an isomerization reaction is catalyzed, selected from the group consisting of molecular beacons, small molecules, fluorophores, chemophores, ionophores, radio-isotopes, photophores, peptides, proteins, enzymes, antibodies, nucleic acids, and enzymatic nucleic acids; L1 and L2 each represent a linker which can be the same or different and which can be present or absent; X1 and X2 represent an atom, compound, or molecule that can be the same or different, and “-” represents a chemical bond.

In another embodiment, the reaction catalyzed by the enzymatic nucleic acid component of the nucleic acid sensor or nucleic acid sensor molecule with the reporter molecule of the invention features catalytic activity, for example, cleavage activity, ligation activity, isomerization activity, phosphorylation activity, dephosphorylation activity, amplification activity, and/or polymerase activity.

The invention also features a method comprising:(a) contacting a nucleic acid sensor molecule comprising an enzymatic nucleic acid component and one or more sensor components, and a reporter molecule with a system under conditions suitable for the enzymatic nucleic acid component of the nucleic acid sensor molecule to attach at least a portion of the reporter molecule to the nucleic acid sensor molecule in the presence of a target signaling agent; and (b) assaying for the attachment of the reporter molecule to the nucleic acid sensor molecule.

In another embodiment, the invention features a method comprising:(a) contacting a nucleic acid sensor molecule comprising an enzymatic nucleic acid component and one or more sensor components, and a reporter molecule with a system under conditions suitable for the enzymatic nucleic acid component of the nucleic acid sensor molecule to isomerize at least a portion of the reporter molecule in the presence of a target signaling agent; and (b) assaying for the isomerization reaction.

In yet another embodiment, the invention features a method comprising: (a) contacting a nucleic acid sensor molecule comprising an enzymatic nucleic acid component and one or more sensor components, and a reporter molecule with a system under conditions suitable for the enzymatic nucleic acid component of the nucleic acid sensor molecule to phosphorylate a non-oligonucleotide-based portion of the reporter molecule in the presence of a target signaling agent; and (b) assaying for the phosphorylation reaction.

In still another embodiment, the invention features a method comprising: (a) contacting a nucleic acid sensor molecule comprising an enzymatic nucleic acid component and one or more sensor components, and a reporter molecule with a system under conditions suitable for the enzymatic nucleic acid component of the nucleic acid sensor molecule to dephosphorylate a non-oligonucleotide-based portion of the reporter molecule in the presence of a target signaling agent; and (b) assaying for the dephosphorylation reaction.

In any of the above-described inventive methods, the system can be an in vitro system. The in vitro system can be, for example, a sample, such as a biological sample, from an organism, mammal, or patient, preferably a human.

In any of the above-described inventive methods, the enzymatic nucleic acid component of said nucleic acid sensor molecule can be a hammerhead, hairpin, inozyme, G-cleaver, Zinzyme, RNase P EGS nucleic acid and Amberzyme motif. Also, in any of the above-described inventive methods, the enzymatic nucleic acid component of said nucleic acid sensor molecule can be a DNAzyme.

In any of the above-described methods, the detection of a chemical reaction is indicative of the presence of the target signaling molecule in the system. In any of the above-described methods, the absence of a chemical reaction is indicative of the system lacking the target signaling molecule.

In one embodiment, the reporter molecule of the instant invention is selected from the group consisting of molecular beacons, small molecules, fluorophores, chemophores, ionophores, radio-isotopes, photophores, peptides, proteins, enzymes, antibodies, nucleic acids, and enzymatic nucleic acids or a combination thereof (see for example in Singh et al., 2000, Biotech., 29, 344; Lizardi et al., U.S. Pat. Nos. 5,652,107 and 5,118,801).

Using such reporter molecules and others known in the art, the detectable response of the instant invention can be monitored by, for example, a change in fluorescence, color change, UV absorbance, phosphorescence, pH, optical rotation, isomerization, polymerization, temperature, mass, capacitance, resistance, and emission of radiation.

Detection of the target signaling event via the chemical reaction or the change in activity or physical properties of the reporter molecule can be assayed by methods discussed herein and others known in the art. Amplification of the target signaling event via the chemical reaction or the change in activity or physical properties of the reporter molecule is accomplished by methods discussed herein and known in the art, for example, modulating polymerase activity. Modulation of polymerase activity can increase polymerization in a chemical reaction, for example, a polymerase chain reaction (PCR) system, resulting in amplification of a target signaling molecule or reporter molecule.

The present invention features a nucleic acid-based sensor molecule comprising an enzymatic nucleic acid component and one or more sensor components. The nucleic acid sensor molecule is selected for having catalytic activity only through interaction with a target signaling agent, such that in response to an interaction of the target signaling agent with at least one sensor component, the enzymatic portion of the nucleic acid sensor molecule catalyzes a chemical reaction.

In one embodiment, the nucleic acid sensor molecule comprises an enzymatic nucleic acid component and one or more sensor components, wherein the enzymatic nucleic acid component and sensor component(s) are distinct moieties.

In one embodiment, the nucleic acid sensor molecule comprises an enzymatic nucleic acid component and one or more sensor components, wherein distinct enzymatic nucleic acid component and sensor component(s) are joined by a linker region. Thus, in one embodiment, a linker region joins one or more enzymatic nucleic acid components to one or more sensor components in the nucleic acid sensor molecules of the instant invention.

As discussed above, the chemical reaction carried out by the nucleic acid sensor molecule can comprise a reaction in which a reporter molecule or a portion of a reporter molecule becomes covalently attached to the nucleic acid sensor molecule. Thus, in another embodiment, the nucleic acid sensor molecule comprises an enzymatic nucleic acid component and one or more sensor components, wherein distinct enzymatic nucleic acid component and sensor component(s) are joined by a covalent bond. In one embodiment, the chemical reaction carried out by the nucleic acid sensor molecule comprises a reaction in which a reporter molecule becomes covalently attached to the nucleic acid sensor molecule that is immobilized on a solid support or surface. Suitable solid surfaces include silicon-based chips, silicon-based beads, controlled pore glass, polystyrene, and cross-linked polystyrene nitrocellulose, biotin, plastics, metals and polyethylene films.

In another embodiment, the nucleic acid sensor molecule comprises an enzymatic nucleic acid component and one or more sensor components, wherein a sensor component of a nucleic acid sensor molecule of the instant invention is an integral part of the enzymatic nucleic acid component of the nucleic acid sensor molecule. Specifically, for example, one or more sensor components of a nucleic acid sensor molecule shares sequence with the enzymatic nucleic acid component of the nucleic acid sensor molecule and is necessary for the activity of the enzymatic nucleic acid component. The sensor component can also be part of the enzymatic nucleic acid component of the nucleic acid sensor molecule.

In the presence of a target signaling molecule, the sensor component activates or facilitates a chemical reaction. Alternatively, in the presence of a target signaling molecule, the sensor component inhibits a chemical reaction from taking place.

In other embodiments, the invention features the use of at least one reporter molecule, at least one target signaling molecule, and a nucleic acid sensor molecule which is comprised of an enzymatic nucleic acid component joined by a linker to one or more sensor components, where a sensor component, for example, is complementary to one or more sequences within the enzymatic nucleic acid component. The ability of the enzymatic nucleic acid component, in the nucleic acid sensor or nucleic acid sensor molecule, to catalyze a reaction is inhibited by the interaction of one or more sensor components. However, in the presence of one or more distinct target signaling molecules, the sensor component interacts with its respective target signaling molecule preferentially, allowing the nucleic acid sensor molecule to interact with a reporter molecule to catalyze a reaction. A catalytic reaction then takes place on the reporter molecule, for example, cleavage or ligation of the reporter molecule, the rate of which can then be measured by standard assays described herein and otherwise well known in the art.

In another embodiment, the invention features a method for the detection and/or amplification of specific target signaling molecules in a system using at least one reporter molecule, at least one target signaling molecule, and a nucleic acid sensor molecule which comprises an enzymatic nucleic acid component and at least one separate sensor component, where the sensor component or components interacts with one or more sequences within the nucleic acid sensor molecule. The ability of the enzymatic nucleic acid, in the nucleic acid sensor molecule, to catalyze a reaction is inhibited by the interaction of at least one sensor component. However, in the presence of a target signaling molecule, the sensor component preferentially interacts with the enzymatic nucleic acid component, which allows the nucleic acid sensor molecule to interact with a reporter molecule and become functional. A catalytic reaction then takes place on the reporter molecule, for example, cleavage or ligation of the reporter molecule, the rate of which can then be measured by standard assays described herein and otherwise well known in the art.

In one embodiment, the invention features a method for the detection and/or amplification of a specific target signaling molecule in a system using at least one reporter molecule, at least one target signaling molecule, and a nucleic acid sensor molecule which comprises an enzymatic nucleic acid component. The nucleic acid sensor molecule is selected for having catalytic activity only through interaction with the target signaling molecule. In the absence of the target signaling molecule, the nucleic acid sensor molecule is inactive. In the presence of a target signaling molecule the nucleic acid sensor molecule adopts an active conformation and become functional. A catalytic reaction then takes place on the reporter molecule, for example, cleavage or ligation of the reporter molecule, the rate of which is measured by standard assays discussed herein and well known in the art. Alternatively, the nucleic acid sensor molecule can be selected to be inhibited through interaction with the target signaling molecule, such that interaction with the target causes the nucleic acid sensor molecule to adopt an inactive conformation and become non-active.

Thus in one embodiment, the present invention features a method comprising: (a) contacting a nucleic acid sensor molecule which comprises (i) an enzymatic nucleic acid component comprising a substrate binding region and a catalytic region; and (ii) a sensor component comprising a nucleic acid sequence that upon interacting with a complementary sequence in the enzymatic nucleic acid component inhibits the activity of the enzymatic nucleic acid component, and a reporter molecule comprising a nucleic acid sequence complementary to the substrate binding region of the enzymatic nucleic acid component of the nucleic acid sensor molecule, with a system under conditions suitable for the enzymatic nucleic acid component of the nucleic acid sensor molecule to catalyze cleavage of the reporter molecule in the presence of a target signaling molecule; and (b) assaying for the cleavage reaction of (a).

In one embodiment of the inventive method, the cleavage of the reporter molecule is indicative of the presence of the target signaling molecule in the system. The absence of cleavage of the reporter molecule is indicative of the system lacking the target signaling molecule.

In another embodiment, the present invention features a method comprising:(a) contacting a nucleic acid sensor molecule which comprises (i) an enzymatic nucleic acid component comprising a substrate binding region and a catalytic region; and (ii) a sensor component comprising a nucleic acid sequence that upon interacting with a complementary sequence in the enzymatic nucleic acid component inhibits the activity of the enzymatic nucleic acid component, and a reporter molecule comprising a nucleic acid sequence complementary to the substrate binding region of the enzymatic nucleic acid component of the nucleic acid sensor molecule, with a system under conditions suitable for the enzymatic nucleic acid component of the nucleic acid sensor molecule to catalyze a ligation reaction involving the reporter molecule in the presence of a target signaling molecule; and (b) assaying for the ligation reaction in (a).

In one embodiment of the inventive method, the ligation reaction causes at least a portion of the reporter molecule to be attached to the nucleic acid sensor molecule. In another embodiment, the ligation reaction causes at least a portion of the reporter molecule to be attached to a separate molecule. Also, in one embodiment of the inventive method, the ligation of the reporter molecule is indicative of the presence of the target signaling molecule in the system. The absence of ligation of the reporter molecule is indicative of the system lacking the target signaling molecule.

In any of the above-described inventive methods, the system can be an in vitro system. The in vitro system can be a sample, such as a biological sample, derived from, for example, an organism, mammal, or patient.

In another embodiment, one or more nucleic acid sensor molecules are attached to a solid support, for example, a silicon-based surface. Each nucleic acid sensor molecule can be attached via one of its termini by a spacer molecule to allow the nucleic acid sensor molecule to adopt the appropriate conformations without hindrance from the underlying solid support. A test mixture is contacted with one or more nucleic acid sensor molecules, and the mixture is contacted with the solid support. Measurement of a signal generated by the nucleic acid sensor molecule in response to interaction with a target signaling molecule at each address of the array reveals the concentration of each target signaling molecule in the test mixture.

In any of the above methods, the enzymatic nucleic acid component of said nucleic acid sensor molecule can be a hammerhead, hairpin, inozyme, G-cleaver, Zinzyme, RNase P, EGS nucleic acid, or Amberzyme motif.

In any of the above methods, the enzymatic nucleic acid component of said nucleic acid sensor molecule can be a DNAzyme.

In any of the above methods, the reporter molecule can comprise a detectable label selected from the group consisting of chromogenic substrate, fluorescent labels, chemiluminescent labels, radioactive labels, and the like.

In any of the above methods, the reporter molecule can be immobilized on a solid support, preferably comprising silicon-based chips, silicon-based beads, controlled pore glass, polystyrene, cross-linked polystyrene, nitrocellulose, biotin, plastics, metals and polyethylene films.

In one embodiment of the invention, the sensor component of the nucleic acid sensor molecule is RNA, DNA, analog of RNA or analog of DNA.

In another embodiment, the sensor component of the nucleic acid sensor molecule is covalently attached to the nucleic acid sensor molecule by a linker. Suitable linkers include, for example, one or more nucleotides, abasic moiety, polyether, polyamine, polyamide, peptide, carbohydrate, lipid, and polyhydrocarbon compounds, and any combination thereof.

In another embodiment, the sensor component of the nucleic acid sensor molecule is not covalently attached to the nucleic acid sensor molecule.

The present invention also provides kits for the detection of particular targets in test mixtures or biological fluids. The kit comprises separate components containing solutions of a nucleic acid sensor molecule specific for a particular target signaling agent, and containing solutions of the appropriate reporter molecules. In some embodiments, the kit comprises a solid support to which is attached the nucleic acid sensor molecule to the particular target. In further embodiments, the kit further comprises a component containing a standardized solution of the target. With this solution, it is possible for the user of the kit to prepare a graph or table of the detectable signal (for example, fluorescence units vs. target concentration); this table or graph is then used to determine the concentration of the target in the test mixture. Devices that automate the manipulation of such kits, perform the repeated function of the kits, combine various steps of kits, or that generate data from the kits are further contemplated by the instant invention.

In one embodiment, the nucleic acid sensor molecules (allozymes) of the invention are used for in vivo applications, for example in vivo ELISA for drug screening. In vivo ELISA is essentially equivalent to western blot analysis. An allozyme specific to an analyte, for example a drug, drug analog, or drug metabolite etc., can be constitutively expressed along with green fluorescent protein (GFP). The allozyme is designed such that when activated it cleaves GFP mRNA thus inhibiting GFP expression. In the presence of an analyte, the GFP signal would not be observed and in the absence of the analyte, full expression of GFP would be achieved. Thus, by monitoring GFP expression the analyte concentration (e.g. protein expression) can be calculated. Similarly in vivo drug screening can be achieved using a similar system. This system would give direct IC50 and EC50 values.

In another non-limiting example, an allozyme can be activated by a predetermined protein, peptide, or mutant polypeptide that causes the allozyme to inhibit the expression of the gene encoding the protein, peptide, or mutant polypeptide, by, for example, cleaving RNA encoded by the gene. In this non-limiting example, the allozyme acts as a decoy to inhibit the function of the protein, peptide, or mutant polypeptide and also inhibit the expression of the protein, peptide, or mutant polypeptide once activated by the protein, peptide, or mutant polypeptide.

Several in vitro selection (evolution) strategies (Orgel, 1979, Proc. R. Soc. London, B 205, 435) have been used to evolve new nucleic acid catalysts capable of catalyzing cleavage and ligation of phosphodiester linkages (Joyce, 1989, Gene, 82, 83-87; Beaudry et al., 1992, Science 257, 635-641; Joyce, 1992, Scientific American 267, 90-97; Breaker et al., 1994, TIBTECH 12, 268; Bartel et al.,1993, Science 261:1411-1418; Szostak, 1993, TIBS 17, 89-93; Kumar et al., 1995, FASEB J., 9, 1183; Breaker, 1996, Curr. Op. Biotech., 7, 442; Santoro et al., 1997, Proc. Natl. Acad. Sci., 94, 4262; Tang et al., 1997, RNA 3, 914; Nakamaye & Eckstein, 1994, supra; Long & Uhlenbeck, 1994, supra; Ishizaka et al., 1995, supra; Vaish et al., 1997, Biochemistry 36, 6495; Kuwabara et al., 2000, Curr. Opin. Chem. Biol., 4, 669) all of these are incorporated by reference herein). Each can catalyze a series of reactions including the hydrolysis of phosphodiester bonds in trans (and thus can cleave other RNA molecules) under physiological conditions.

There are several classes of enzymatic nucleic acids that are presently known. Each can catalyze the hydrolysis of RNA phosphodiester bonds in trans (and thus can cleave other RNA molecules) under physiological conditions. In general, enzymatic nucleic acids act by first binding to a target. Such binding occurs, for example, through the interaction of the target RNA with one or more target binding portions of the enzymatic nucleic acid, wherein the target RNA and substrate binding portion complex is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its function, such as its ability to direct synthesis of an encoded protein. After an enzymatic nucleic acid has bound and cleaved its RNA target, it is released from that RNA to search for another target and can repeatedly bind and cleave new targets. Thus, a single enzymatic nucleic acid molecule is able to cleave many molecules of target RNA. In addition, the enzymatic nucleic acid molecule is a highly specific inhibitor of gene expression, with the specificity of inhibition depending not only on the base-pairing mechanism of binding to the target RNA, but also on the mechanism of target RNA cleavage. Single mismatches, or base-substitutions, near the site of cleavage can completely eliminate catalytic activity of an enzymatic nucleic acid.

In one embodiment, the invention provides a method for producing a class of nucleic acid-based diagnostic agents that exhibit a high degree of specificity for the target signaling molecule, such as a drug, drug analog, of drug metabolite.

In another embodiment, the invention features a method of detecting drug target signaling molecules or signaling agents in both in vitro and in vivo applications. In vitro diagnostic applications can comprise both solid support based and solution based chip, multichip-array, micro-well plate, and micro-bead derived applications as are commonly known in the art, as weel as other applications discussed herein. In vivo diagnostic applications can include but are not limited to cell culture and animal model based applications.

By “signaling agent” or “target signaling agent” is meant a chemical or physical entity capable of interacting with a nucleic acid sensor molecule, specifically a sensor component of a nucleic acid sensor molecule, in a manner that causes the nucleic acid sensor molecule to be active. The interaction of the signaling agent with a nucleic acid sensor molecule may result in modification of the enzymatic nucleic acid component of the nucleic acid sensor molecule via chemical, physical, topological, or confomiational changes to the structure of the molecule, such that the activity of the enzymatic nucleic acid component of the nucleic acid sensor molecule is modulated, for example is activated or deactivated. Signaling agents of the instant invention can comprise target signaling molecules such as drug compounds that are generally known to be associated with substance abuse, for both recreational, mood-altering, or performance enhancing use. Such compounds can be assayed in mammalian subjects, including human and animal subjects, such as in testing athletes, thoroughbred horses and greyhound dogs.

By “enzymatic nucleic acid” is meant a nucleic acid molecule capable of catalyzing (altering the velocity and/or rate of) a variety of reactions including the ability to repeatedly cleave other separate nucleic acid molecules (endonuclease activity) or ligate other separate nucleic acid molecules (ligation activity) in a nucleotide base sequence-specific manner. Additional reactions amenable to nucleic acid sensor molecules include but are not limited to phosphorylation, dephosphorylation, isomerization, helicase activity, polymerization, transesterification, hydration, hydrolysis, alkylation, dealkylation, halogenation, dehalogenation, esterification, desterification, hydrogenation, dehydrogenation, saponification, desaponification, amination, deamination, acylation, deacylation, glycosylation, deglycosylation, silation, desilation, hydroboration, epoxidation, peroxidation, carboxylation, decarboxylation, substitution, elimination, oxidation, and reduction reactions on both small molecules and macromolecules.

Such a molecule with endonuclease and/or ligation activity can have complementarity in a substrate binding region to a specified gene target, and also has an enzymatic activity that specifically cleaves and/or ligates RNA or DNA in that target. That is, the nucleic acid molecule with endonuclease and/or ligation activity is able to intramolecularly or intermolecularly cleave and/or ligate RNA or DNA and thereby inactivate or activate a target RNA or DNA molecule. This complementarity functions to allow sufficient hybridization of the enzymatic RNA molecule to the target RNA or DNA reporter molecule to allow the cleavage/ligation to occur. 100% complementarity is preferred, but complementarity as low as 50-75% can also be useful in this invention.

In addition, nucleic acid sensor molecule can perform other reactions, including those mentioned above, selectively on both small molecule and macromolecular substrates, though specific interaction of the nucleic acid sensor molecule sequence with the desired substrate molecule via hydrogen bonding, electrostatic interactions, and Van der Waals interactions.

The nucleic acids can be modified at the base, sugar, and/or phosphate groups. The term enzymatic nucleic acid is used interchangeably with phrases such as ribozymes, catalytic RNA, enzymatic RNA, catalytic DNA, catalytic oligonucleotides, nucleozyme, DNAzyme, RNA enzyme, endoribonuclease, endonuclease, minizyme, leadzyme, oligozyme, finderon or DNA enzyme. All of these terminologies describe nucleic acid molecules with enzymatic activity.

There are several different structural motifs of enzymatic nucleic acid molecules that catalyze cleavage/ligations reaction, including but not limited to hammerhead motif, hairpin motif, hepatitis delta virus motif, G-cleaver motif, Amberzyme motif, inozyme motif, and Zinzyme motif Other motifs can be evolved using in vitro or in vivo selection techniques.

By “substrate binding arm” or “substrate binding domain” or “substrate binding region” is meant that portion or region of a nucleic acid sensor molecule which is able to interact, for example, via complementarity (i.e., able to base-pair with), with a portion of its substrate or reporter. Preferably, such complementarity is 100%, but can be less if desired. For example, as few as 10 bases out of 14 can be base-paired (see for example Werner and Uhlenbeck, 1995, Nucleic Acids Research, 23, 2092-2096; Hammann et al., 1999, Antisense and Nucleic Acid Drug Dev., 9, 25-31). That is, these arms contain sequences within a nucleic acid sensor molecule which are intended to bring the nucleic acid sensor molecule and the reporter molecule, for example RNA, together through complementary base-pairing interactions. The nucleic acid sensor molecule of the invention can have binding arms that are contiguous or non-contiguous and can be of varying lengths. The length of the binding arm(s) are preferably greater than or equal to four nucleotides and of sufficient length to stably interact with the target reporter sequence. Preferably, the binding arm(s) are 12-100 nucleotides in length. More preferably, the binding arms are 14-24 nucleotides in length (see, for example, Werner and Uhlenbeck, supra; Hamman et al., supra; Hampel et al., EP0360257; Berzal-Herrance et al., 1993, EMBO J., 12, 2567-73). If two binding arms are chosen, the design is such that the length of the binding arms are symmetrical (i.e., each of the binding arms is of the same length; e.g., five and five nucleotides, or six and six nucleotides, or seven and seven nucleotides long) or asymmetrical (i.e., the binding arms are of different length; e.g., six and three nucleotides; three and six nucleotides long; four and five nucleotides long; four and six nucleotides long; four and seven nucleotides long; and the like).

By “enzymatic portion” or “catalytic domain” is meant that portion or region of the nucleic acid sensor molecule essential for catalyzing a chemical reaction, such as cleavage of a nucleic acid substrate.

By “system” or “sample” is meant material, in a purified or unpurified form, from biological or non-biological sources, including but not limited to human, animal, plant, bacteria, virus, fungi, soil, water, mechanical devices, circuits, networks, computers, or others that comprises the target signaling agent or target signaling molecule to be detected. As such, nucleic acid sensor molecules and aptamers of the invention can be used to assay target compounds in biologic and non-biologic systems, such as in human and animal subjects or in samples of unidentified materials outside of a biological system.

The “biological system” or “biological sample” as used herein can be a eukaryotic system or a prokaryotic system, for example a bacterial cell, plant cell or a mammalian cell, or of plant origin, mammalian origin, yeast origin, Drosophila origin, or archebacterial origin.

By “reporter molecule” is meant a molecule, such as a nucleic acid sequence (e.g., RNA or DNA or analogs thereof) or peptides and/or other chemical moieties, able to stably interact with the nucleic acid sensor molecule and function as a substrate for the nucleic acid sensor molecule. The reporter molecule can be covalently linked to the nucleic acid sensor molecule or a portion of one of the components of a halfzyme. The reporter molecule can also contain chemical moieties capable of generating a detectable response, including but not limited to, fluorescent, chromogenic, radioactive, enzymatic and/or chemiluminescent or other detectable labels that can then be detected using standard assays known in the art. The reporter molecule can also act as an intermediate in a chain of events, for example, by acting as an amplicon, inducer, promoter, or inhibitor of other events that can act as second messengers in a system.

In one embodiment, the reporter molecule of the invention is an oligonucleotide primer, template, or probe, which can be used to modulate the amplification of additional nucleic acid sequences, for example, sequences comprising reporter molecules, target signaling molecules, effector molecules, inhibitor molecules, and/or additional nucleic acid sensor molecules of the instant invention.

By “sensor component” or “sensor domain” of the nucleic acid sensor molecule is meant, a molecule such as a nucleic acid sequence (e.g., RNA or DNA or analogs thereof), peptide, or other chemical moiety which can interact with one or more regions of a target signaling agent or more than one target signaling agents, and which interaction causes the enzymatic nucleic acid component of the nucleic acid sensor molecule to modulate, such as inhibit or activate, the catalytic activity of the nucleic acid sensor molecule. In the presence of a signaling agent, the ability of the sensor component, for example, to modulate the catalytic activity of the enzymatic nucleic acid component is inhibited or diminished. The sensor component can comprise recognition properties relating to chemical or physical signals capable of modulating the enzymatic nucleic acid component via chemical or physical changes to the structure of the nucleic acid sensor molecule. The sensor component can also be derived from a nucleic acid sequence that is obtained through in vitro or in vivo selection techniques as are know in the art. Alternately, the sensor component can be derived from a nucleic acid molecule (aptamer) which is evolved to bind to a nucleic acid sequence within a target nucleic acid molecule. Such sequences or “aptamers” can be designed to bind a specific protein, peptide, nucleic acid, co-factor, metabolite, drug, or other small molecule with varying affinity. The sensor component can be covalently linked to the nucleic acid sensor molecule, or can be non-covalently associated. A person skilled in the art will recognize that all that is required is that the sensor component is able to selectively inhibit the activity of the nucleic acid sensor molecule.

“Complementarity” refers to the ability of a nucleic acid to form hydrogen bond(s) with another RNA sequence by either traditional Watson-Crick or other non-traditional types. In reference to the nucleic molecules of the present invention, the binding free energy for a nucleic acid molecule with its target or complementary sequence is sufficient to allow the relevant function of the nucleic acid to proceed, e.g., enzymatic nucleic acid cleavage, ligation, isomerization, phosphorylation, or dephosphorylation. Determination of binding free energies for nucleic acid molecules is well known in the art (see, e.g., Turner et al., 1987, CSH Symp. Quant. Biol. LII pp.123-133; Frier et al., 1986, Proc. Nat. Acad. Sci. USA 83:9373-9377; Turner et al., 1987, J. Am. Chem. Soc. 109:3783-3785). A percent complementarity indicates the percentage of contiguous residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence.

By “alkyl” group is meant a saturated aliphatic hydrocarbon, including straight-chain, branched-chain, and cyclic alkyl groups. Preferably, the alkyl group has 1 to 12 carbons. More preferably it is a lower alkyl of from 1 to 7 carbons, more preferably 1 to 4 carbons. The alkyl group can be substituted or unsubstituted. When substituted the substituted group(s) are preferably, hydroxyl, cyano, alkoxy, ═O, ═S, NO₂ or N(CH₃)₂, amino, or SH. The term also includes alkenyl groups which are unsaturated hydrocarbon groups containing at least one carbon-carbon double bond, including straight-chain, branched-chain, and cyclic groups. Preferably, the alkenyl group has 1 to 12 carbons. More preferably it is a lower alkenyl of from 1 to 7 carbons, more preferably 1 to 4 carbons. The alkenyl group can be substituted or unsubstituted. When substituted the substituted group(s) can be preferably, hydroxyl, cyano, alkoxy, ═O, ═S, NO₂, halogen, N(CH₃)₂, amino, or SH. The term “alkyl” also includes alkynyl groups which have an unsaturated hydrocarbon group containing at least one carbon-carbon triple bond, including straight-chain, branched-chain, and cyclic groups. Preferably, the alkynyl group has 1 to 12 carbons. More preferably it is a lower alkynyl of from 1 to 7 carbons, more preferably 1 to 4 carbons. The alkynyl group can be substituted or unsubstituted. When substituted the substituted group(s) is preferably, hydroxyl, cyano, alkoxy, ═O, ═S, NO₂ or N(CH₃)₂, amino or SH. Such alkyl groups can also include aryl, alkylaryl, carbocyclic aryl, heterocyclic aryl, amide and ester groups. An “aryl” group refers to an aromatic group which has at least one ring having a conjugated p electron system and includes carbocyclic aryl, heterocyclic aryl and biaryl groups, all of which can be optionally substituted. The preferred substituent(s) of aryl groups are halogen, trihalomethyl, hydroxyl, SH, OH, cyano, alkoxy, alkyl, alkenyl, alkynyl, and amino groups. An “alkylaryl” group refers to an alkyl group (as described above) covalently joined to an aryl group (as described above). Carbocyclic aryl groups are groups wherein the ring atoms on the aromatic ring are all carbon atoms. The carbon atoms are optionally substituted. Heterocyclic aryl groups are groups having from 1 to 3 heteroatoms as ring atoms in the aromatic ring and the remainder of the ring atoms are carbon atoms. Suitable heteroatoms include oxygen, sulfur, and nitrogen, and include furanyl, thienyl, pyridyl, pyrrolyl, N-lower alkyl pyrrolo, pyrimidyl, pyrazinyl, imidazolyl and the like, all optionally substituted. An “amide” refers to an —C(O)—NH—R, where R is either alkyl, aryl, alkylaryl or hydrogen. An “ester” refers to an —C(O)—OR′, where R is either alkyl, aryl, alkylaryl or hydrogen.

By “nucleotide” is meant a heterocyclic nitrogenous base in N-glycosidic linkage with a phosphorylated sugar. Nucleotides are recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein). There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra). By “modified bases” in this aspect is meant nucleotide bases other than adenine, guanine, cytosine and uracil at 1′ position or their equivalents; such bases can be used at any position, for example, within the catalytic core of an nucleic acid sensor molecule and/or in the substrate-binding regions of the nucleic acid molecule.

By “nucleoside” is meant a heterocyclic nitrogenous base in N-glycosidic linkage with a sugar. Nucleosides are recognized in the art to include natural bases (standard), and modified bases well known in the art. Such bases are generally located at the 1′ position of a nucleoside sugar moiety. Nucleosides generally comprise a base and sugar group. The nucleosides can be unmodified or modified at the sugar, and/or base moiety, (also referred to interchangeably as nucleoside analogs, modified nucleosides, non-natural nucleosides, non-standard nucleosides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein). There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethylunidine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra). By “modified bases” in this aspect is meant nucleoside bases other than adenine, guanine, cytosine and uracil at 1′ position or their equivalents; such bases can be used at any position, for example, within the catalytic core of an nucleic acid sensor molecule and/or in the substrate-binding regions of the nucleic acid molecule.

By “unmodified nucleotide” is meant a nucleotide with one of the bases adenine, cytosine, guanine, thymine, uracil joined to the 1′ carbon of beta-D-ribo-furanose.

By “modified nucleotide” is meant a nucleotide that contains a modification in the chemical structure of an unmodified nucleotide base, sugar and/or phosphate.

By “unmodified nucleoside” is meant a nucleoside with one of the bases adenine, cytosine, guanine, thymine, uracil joined to the 1′ carbon of beta-D-ribo-furanose.

By “modified nucleoside” is meant a nucleotide that contains a modification in the chemical structure of an unmodified nucleoside base or sugar.

By “Inozyme” or “NCH” motif or configuration is meant, an enzymatic nucleic acid molecule comprising a motif as is generally described as NCH Rz in Ludwig et al., International PCT Publication No. WO 98/58058 and U.S. patent application Ser. No. 08/878,640, which is herein incorporated by reference in its entirety including the drawings. Inozymes possess endonuclease activity to cleave RNA substrates having a cleavage triplet NCH/, where N is a nucleotide, C is cytidine and H is adenosine, uridine or cytidine, and / represents the cleavage site. Inozymes can also possess endonuclease activity to cleave RNA substrates having a cleavage triplet NCN/, where N is a nucleotide, C is cytidine, and / represents the cleavage site

By “G-cleaver” motif or configuration is meant, an enzymatic nucleic acid molecule comprising a motif as is generally described in Eckstein et al., U.S. Pat. No. 6,127,173, which is herein incorporated by reference in its entirety including the drawings, and in Kore et al., 1998, Nucleic Acids Research 26, 4116-4120. G-cleavers possess endonuclease activity to cleave RNA substrates having a cleavage triplet NYN/, where N is a nucleotide, Y is uridine or cytidine and / represents the cleavage site. G-cleavers can be chemically modified.

By “zinzyme” motif or configuration is meant, an enzymatic nucleic acid molecule comprising a motif as is generally described in Beigelman et al., International PCT publication No. WO 99/55857 and U.S. patent application Ser. No. 09/918,728, which is herein incorporated by reference in its entirety including the drawings. Zinzymes possess endonuclease activity to cleave RNA substrates having a cleavage triplet including but not limited to, YG/Y, where Y is uridine or cytidine, and G is guanosine and / represents the cleavage site. Zinzymes can be chemically modified to increase nuclease stability through various substitutions, including substituting 2′-O-methyl guanosine nucleotides for guanosine nucleotides. In addition, differing nucleotide and/or non-nucleotide linkers can be used to substitute the 5′-gaaa-2′ loop of the motif. Zinzymes represent a non-limiting example of an enzymatic nucleic acid molecule that does not require a ribonucleotide (2′-OH) group within its own nucleic acid sequence for activity.

By “amberzyme” motif or configuration is meant, an enzymatic nucleic acid molecule comprising a motif as is generally described in Beigelman et al., International PCT publication No. WO 99/55857 and U.S. patent application Ser. No. 09/476,387, which is herein incorporated by reference in its entirety including the drawings. Amberzymes possess endonuclease activity to cleave RNA substrates having a cleavage triplet NG/N, where N is a nucleotide, G is guanosine, and / represents the cleavage site. Amberzymes can be chemically modified to increase nuclease stability. In addition, differing nucleoside and/or non-nucleoside linkers can be used to substitute the 5′-gaaa-3′ loops of the motif. Amberzymes represent a non-limiting example of an enzymatic nucleic acid molecule that does not require a ribonucleotide (2′-OH) group within its own nucleic acid sequence for activity.

By ‘DNAzyme’ is meant, an enzymatic nucleic acid molecule that does not require the presence of a 2′-OH group within its own nucleic acid sequence for activity. In particular embodiments, the enzymatic nucleic acid molecule can have an attached linker or linkers or other attached or associated groups, moieties, or chains containing one or more nucleotides with 2′-OH groups. DNAzymes can be synthesized chemically or expressed endogenously in vivo, by means of a single stranded DNA vector or equivalent thereof. Non-limiting examples of DNAzymes are generally reviewed in Usman et al., U.S. Pat. No. 6,159,714, which is herein incorporated by reference in its entirety including the drawings; Chartrand et al., 1995, NAR 23, 4092; Breaker et al., 1995, Chem. Bio. 2, 655; Santoro et al., 1997, PNAS 94, 4262; Breaker, 1999, Nature Biotechnology, 17, 422-423; and Santoro et. al., 2000, J. Am. Chem. Soc., 122, 2433-39. The “10-23” DNAzyme motif is one particular type of DNAzyme that was evolved using in vitro selection as generally described in Joyce et al., U.S. Pat. No. 5,807,718 and Santoro et al., supra. Additional DNAzyme motifs can be selected for using techniques similar to those described in these references, and hence, are within the scope of the present invention.

By “sufficient length” is meant an oligonucleotide of length sufficient to provide the intended function (such as binding) under the expected condition. For example, a binding arm of the enzymatic nucleic acid component of the nucleic acid sensor molecule should be of “sufficient length” to provide stable binding to the reporter molecule under the expected reaction conditions and environment to catalyze a reaction. In a further example, the sensor domain of the nucleic acid sensor molecule should be of sufficient length to interact with a target nucleic acid molecule in a manner that would cause the nucleic acid sensor to be active.

By “stably interact” is meant interaction of the oligonucleotides with target nucleic acid (e.g., by forming hydrogen bonds with complementary nucleotides in the target under physiological conditions) that is sufficient for the intended purpose (e.g., cleavage of target RNA by an enzyme).

By “nucleic acid molecule” as used herein is meant a molecule comprising nucleotides. The nucleic acid can be single, double, or multiple stranded and can comprise modified or unmodified nucleotides or non-nucleotides or various mixtures and combinations thereof. Nucleic acid molecules shall include oligonucleotides, ribozymes, DNAzymes, templates, and primers.

By “oligonucleotide” is meant a nucleic acid molecule comprising a stretch of three or more nucleotides.

In one embodiment, the linker region, when present in the nucleic acid sensor molecule and/or reporter molecule is further comprised of nucleotide, non-nucleotide chemical moieties or combinations thereof. Non-limiting examples of non-nucleotide chemical moieties can include ester, anhydride, amide, nitrile, and/or phosphate groups.

In another embodiment, the non-nucleotide linker is as defined herein. The term “non-nucleotide” as used herein include either abasic nucleotide, polyether, polyamine, polyamide, peptide, carbohydrate, lipid, or polyhydrocarbon compounds. Specific examples include those described by Seela and Kaiser, Nucleic Acids Res. 1990, 18:6353 and Nucleic Acids Res. 1987, 15:3113; Cload and Schepartz, J. Am. Chem. Soc. 1991, 113:6324; Richardson and Schepartz, J. Am. Chem. Soc. 1991, 113:5109; Ma et al., Nucleic Acids Res. 1993, 21:2585 and Biochemistry 1993, 32:1751; Durand et al., Nucleic Acids Res. 1990, 18:6353; McCurdy et al., Nucleosides & Nucleotides 1991, 10:287; Jschke et al., Tetrahedron Lett. 1993, 34:301; Ono et al., Biochemistry 1991, 30:9914; Arnold et al., International Publication No. WO89/02439; Usman et al., International Publication No. WO 95/06731; Dudycz et al., International Publication No. WO 95/11910 and Ferentz and Verdine, J. Am. Chem. Soc. 1991, 113:4000, all hereby incorporated by reference herein. Thus, in a preferred embodiment, the invention features an nucleic acid sensor molecule of the invention having one or more non-nucleotide moieties, and having enzymatic activity to perform a chemical reaction, for example to cleave an RNA or DNA molecule.

By “cap structure” is meant chemical modifications which have been incorporated at either terminus of the oligonucleotide (see for example Wincott et al., WO 97/26270, incorporated by reference herein). These terminal modifications protect the nucleic acid molecule from exonuclease degradation, and can help in delivery and/or localization within a cell. The cap can be present at the 5′-terminus (5′-cap) or at the 3′-terminus (3′-cap) or can be present on both termini. In non-limiting examples: the 5′-cap is selected from the group comprising inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (for more details see Wincott et al., International PCT publication No. WO 97/26270, incorporated by reference herein). In yet another preferred embodiment the 3′-cap is selected from a group comprising, 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Iyer, 1993, Tetrahedron 49, 1925; incorporated by reference herein).

By “abasic” or “abasic nucleotide” is meant sugar moieties lacking a base or having other chemical groups in place of a base at the 1′ position, (for more details see Wincott et al., International PCT publication No. WO 97/26270).

The term “non-nucleotide” refers to any group or compound which can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenine, guanine, cytosine, uracil or thymine. The terms “abasic” or “abasic nucleotide” arc meant to include sugar moieties lacking a base or having other chemical groups in place of a base at the 1′ position, (for more details see Wincott et al., International PCT publication No. WO 97/26270).

By “RNA” is meant a molecule comprising at least one ribonucleotide residue. By “ribonucleotide” or “2′-OH” is meant a nucleotide with a hydroxyl group at the 2′ position of a β-D-ribo-furanose moiety.

By “subject” is meant an organism, which is a donor or recipient of explanted cells or the cells themselves. “Subject” also refers to an organism to which the nucleic acid molecules of the invention can be administered. Preferably, a subject is a mammal or mammalian cells. More preferably, a subject is a human or human cells.

By “enhanced enzymatic activity” is meant to include activity measured in cells and/or in vivo where the activity is a reflection of both the catalytic activity and the stability of the nucleic acid molecules of the invention. In this invention, the product of these properties can be increased in vivo compared to an all RNA enzymatic nucleic acid or all DNA enzyme. In some cases, the individual catalytic activity or stability of the nucleic acid molecule can be decreased (i.e., less than ten-fold), but the overall activity of the nucleic acid molecule is enhanced, in vivo.

By “detectable response” is meant a chemical or physical property that can be measured, including, but not limited to changes in temperature, pH, frequency, charge, capacitance, or changes in fluorescent, chromogenic, colorimetric, radioactive, enzymatic and/or chemiluminescent levels or other properties that can then be detected using standard methods discussed herein and known in the art.

By “predetermined target” is meant a signaling agent or target signaling agent that is chosen to interact with a nucleic acid sensor molecule to generate a detectable response.

Nucleic Acid Molecule Synthesis

The nucleic acid molecules of the invention, including certain nucleic acid sensor molecules, can be synthesized using the methods described in Usman et al., 1987, J. Am. Chein. Soc., 109, 7845; Scaringe et al., 1990, Nucleic Acids Res., 18, 5433; and Wincott et al., 1995, Nucleic Acids Res. 23, 2677-2684 Wincott et al., 1997, Methods Mol. Bio., 74, 59. Such methods make use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 7.5 min coupling step for alkylsilyl protected nucleotides and a 2.5 min coupling step for 2′-O-methylated nucleotides. Table II outlines the amounts and the contact times of the reagents used in the synthesis cycle. Alternatively, syntheses at the 0.2 μmol scale can be done on a 96-well plate synthesizer, such as the PG2100 instrument produced by Protogene (Palo Alto, Calif.) with minimal modification to the cycle. A 33-fold excess (60 μL of 0.11 M=6.6 μmol) of 2′-O-methyl phosphoramidite and a 75-fold excess of S-ethyl tetrazole (60 μL of 0.25 M=15 μmol) can be used in each coupling cycle of 2′-O-methyl residues relative to polymer-bound 5′-hydroxyl. A 66-fold excess (120 μL of 0.11 M=13.2 μmol) of alkylsilyl (ribo) protected phosphoramidite and a 150-fold excess of S-ethyl tetrazole (120 μL of 0.25 M=30 μmol) can be used in each coupling cycle of ribo residues relative to polymer-bound 5′-hydroxyl. Average coupling yields on the 394 Applied Biosystems, Inc. synthesizer, determined by colorimetric quantitation of the trityl fractions, are typically 97.5-99%. Other oligonucleotide synthesis reagents for the 394 Applied Biosystems, Inc. synthesizer include; detritylation solution is 3% TCA in methylene chloride (ABI); capping is performed with 16% N-methyl imidazole in THF (ABI) and 10% acetic anhydride/10% 2,6-lutidine in THF (ABI); oxidation solution is 16.9 mM I₂, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide 0.05 M in acetonitrile) is used.

Cleavage from the solid support and deprotection of the oligonucleotide is typically performed using either a two-pot or one-pot protocol. For the two-pot protocol, the polymer-bound trityl-on oligoribonucleotide is transferred to a 4 mL glass screw top vial and suspended in a solution of 40% aq. methylamine (1 mL) at 65° C. for 10 min. After cooling to −20° C., the supernatant is removed from the polymer support. The support is washed three times with 1.0 mL of EtOH:MeCN:H2O/3:1:1, vortexed and the supernatant is then added to the first supernatant. The combined supernatants, containing the oligoribonucleotide, are dried to a white powder. The base deprotected oligoribonucleotide is resuspended in anhydrous TEA/HF/NMP solution (300 μL of a solution of 1.5 mL N-methylpyrrolidinone, 750 μL TEA and 1 mL TEA.3HF to provide a 1.4 M HF concentration) and heated to 65° C. After 1.5 h, the oligomer is quenched with 1.5 M NH₄HCO₃.

Alternatively, for the one-pot protocol, the polymer-bound trityl-on oligoribonucleotide is transferred to a 4 mL glass screw top vial and suspended in a solution of 33% ethanolic methylamine/DMSO: 1/1 (0.8 mL) at 65° C. for 15 min. The vial is brought to r.t. TEA.3HF (0.1 mL) is added and the vial is heated at 65° C. for 15 min. The sample is cooled at −20° C. and then quenched with 1.5 M NH₄HCO₃. An alternative deprotection cocktail for use in the one pot protocol comprises the use of aqueous methylamine (0.5 ml) at 65° C. for 15 min followed by DMSO (0.8 ml) and TEA.3HF (0.3 ml) at 65° C. for 15 min. A similar methodology can be employed with 96-well plate synthesis formats by using a Robbins Scientific Flex Chem block, in which the reagents are added for cleavage and deprotection of the oligonucleotide.

For anion exchange desalting of the deprotected oligomer, the TEAB solution is loaded onto a Qiagen 500® anion exchange cartridge (Qiagen Inc.) that is prewashed with 50 mM TEAB (10 mL). After washing the loaded cartridge with 50 mM TEAB (10 mL), the RNA is eluted with 2 M TEAB (10 mL) and dried down to a white powder.

For purification of the trityl-on oligomers, the quenched NH₄HCO₃ solution is loaded onto a C-18 containing cartridge that had been prewashed with acetonitrile followed by 50 mM TEAA. After washing the loaded cartridge with water, the RNA is detritylated with 0.5% TFA for 13 min. The cartridge is then washed again with water, salt exchanged with 1 M NaCl and washed with water again. The oligonucleotide is then eluted with 30% acetonitrile. Alternatively, for oligonucleotides synthesized in a 96-well format, the crude trityl-on oligonucleotide is purified using a 96-well solid phase extraction block packed with C18 material, on a Bahdan Automation workstation.

The average stepwise coupling yields are typically >98% (Wincott et al., 1995 Nucleic Acids Res. 23, 2677-2684). Those of ordinary skill in the art will recognize that the scale of synthesis can be adapted as larger or smaller than the example described above including but not limited to 96 well format, all that is important is the ratio of chemicals used in the reaction.

To ensure the quality of synthesis of nucleic acid molecules of the invention, quality control measures are utilized for the analysis of nucleic acid material. Capillary Gel Electrophoresis, for example using a Beckman MDQ CGE instrument, can be ulitized for rapid analysis of nucleic acid molecules, by introducing sample on the short end of the capillary. In addition, mass spectrometry, for example using a PE Biosystems Voyager-DE MALDI instrument, in combination with the Bohdan workstation, can be utilized in the analysis of oligonucleotides, including oligonucleotides synthesized in the 96-well format.

The nucleic acids of the invention can also be synthesized in two parts and annealed to reconstruct the nucleic acid sensor molecules (Chowrira and Burke, 1992 Nucleic Acids Res., 20, 2835-2840). The nucleic acids are also synthesized enzymatically using a variety of methods known in the art, for example as described in Havlina, International PCT publication No. WO 9967413, or from DNA templates using bacteriophage T7 RNA polymerase (Milligan and Uhlenbeck, 1989, Methods Enzymol. 180, 51). Other methods of enzymatic synthesis of the nucleic acid molecules of the invention are generally described in Kim et al., 1995, Biotechniques, 18, 992; Hoffman et al., 1994, Biotechniques, 17, 372; Cazenare et al., 1994, PNAS USA, 91, 6972; Hyman, U.S. Pat. No. 5,436,143; and Karpeisky et al., International PCT publication No. WO 98/28317)

Alternatively, the nucleic acid molecules of the present invention can be synthesized separately and joined together post-synthetically, for example by ligation (Moore et al., 1992, Science 256, 9923; Draper et al., International PCT publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204).

The nucleic acid molecules of the present invention are preferably modified to enhance stability by modification with nuclease resistant groups, for example, 2′-amino, 2′-C-allyl, 2′-flouro, 2′-O-methyl, 2′-H (for a review see Usman and Cedergren, 1992, TIBS 17, 34; Usman et al., 1994, Nucleic Acids Symp. Ser. 31, 163). Nucleic acid sensor molecules are purified by gel electrophoresis using known methods or are purified by high pressure liquid chromatography (HPLC; See Wincott et al., Supra, the totality of which is hereby incorporated herein by reference) and are re-suspended in water.

Optimizing Nucleic Acid Molecule Activity

Synthesizing nucleic acid molecules with modifications (base, sugar and/or phosphate) that prevent their degradation by serum ribonucleases can increase their potency (see e.g., Eckstein et al., International Publication No. WO92/07065; Perrault et al., 1990 Nature 344, 565; Pieken et al., 1991, Science 253, 314; Usman and Cedergren, 1992, Trends in Biochem. Sci. 17, 334; Usman et al., International Publication No. WO93/15187; Rossi et al., International Publication No. WO 91/03162; Sproat, U.S. Pat. No. 5,334,711; and Burgin et al., supra; all of these describe various chemical modifications that can be made to the base, phosphate and/or sugar moieties of the nucleic acid molecules described herein. All these references are incorporated by reference herein. Modifications which enhance their efficacy in cells, and removal of bases from nucleic acid molecules to shorten oligonucleotide synthesis times and reduce chemical requirements are preferably desired.

There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with nuclease resistant groups, for example, 2′-amino, 2′-C-allyl, 2′-flouro, 2′-O-methyl, 2′-H, nucleotide base modifications (for a review see Usman and Cedergren, 1992, TIBS. 17, 34; Usman et al., 1994, Nucleic Acids Symp. Ser. 31, 163; Burgin et al., 1996, Biochemistry, 35, 14090). Sugar modifications of nucleic acid molecules have been extensively described in the art (see Eckstein et al., International Publication PCT No. WO 92/07065; Perrault et al. Nature, 1990, 344, 565-568; Pieken et al. Science, 1991, 253, 314-317; Usman and Cedergren, Trends in Biochem. Sci., 1992, 17, 334-339; Usman et al. International Publication PCT No. WO 93/15187; Sproat, U.S. Pat. No. 5,334,711 and Beigelman et al., 1995, J. Biol. Chem., 270, 25702; Beigelman et al., International PCT publication No. WO 97/26270; Beigelman et al., U.S. Pat. No. 5,716,824; Usman et al., U.S. Pat. No. 5,627,053; Woolf et al., International PCT Publication No. WO 98/13526; Thompson et al., U.S. Ser. No. 60/082,404 which was filed on Apr. 20, 1998; Karpeisky et al., 1998, Tetrahedron Lett., 39, 1131; Earnshaw and Gait, 1998, Biopolymers (Nucleic acid Sciences), 48, 39-55; Verma and Eckstein, 1998, Annu. Rev. Biochem., 67, 99-134; and Burlina et al., 1997, Bioorg. Med. Chem., 5, 1999-2010; all of the references are hereby incorporated by reference herein in their totalities). Such publications describe general methods and strategies to determine the location of incorporation of sugar, base and/or phosphate modifications and the like into nucleic acid sensor molecule molecules without inhibiting catalysis. In view of such teachings, similar modifications can be used as described herein to modify the nucleic acid molecules of the instant invention.

While chemical modification of oligonucleotide internucleotide linkages with phosphorothioate, phosphorothioate, and/or 5′-methylphosphonate linkages improves stability, many of these modifications can cause some toxicity. Therefore when designing nucleic acid molecules the amount of these internucleotide linkages should be minimized. The reduction in the concentration of these linkages should lower toxicity resulting in increased efficacy and higher specificity of these molecules.

Nucleic acid molecules having chemical modifications which maintain or enhance activity are provided. Such nucleic acid is also generally more resistant to nucleases than unmodified nucleic acid. Thus, in the presence of biological fluids, or in cells, the activity can not be significantly lowered. Clearly, nucleic acid molecules must be resistant to nucleases in order to function as effective diagnostic agents, whether utilized in vitro and/or in vivo. Improvements in the synthesis of RNA and DNA (Wincott et al., 1995 Nucleic Acids Res. 23, 2677; Caruthers et al., 1992, Methods in Enzymology 211,3-19; Karpeisky et al., International PCT publication No. WO 98/28317) (incorporated by reference herein) have expanded the ability to modify nucleic acid molecules by introducing nucleotide modifications to enhance their nuclease stability as described above.

In another aspect the nucleic acid molecules comprise a 5′ and/or a 3′-cap structure.

In one embodiment, the invention features modified nucleic acid molecules with phosphate backbone modifications comprising one or more phosphorothioate, phosphorodithioate, methylphosphonate, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications see Hunziker and Leumann, 1995, Nucleic Acid Analogues: Synthesis and Properties, in Modern Synthetic Methods, VCH, 331-417, and Mesmaeker et al., 1994, Novel Backbone Replacements for Oligonucleotides, in Carbohydrate Modifications in Antisense Research, ACS, 24-39. These references are hereby incorporated by reference herein.

In connection with 2′-modified nucleotides as described for the present invention, by “amino” is meant 2′-NH₂ or 2′-O—NH₂, which can be modified or unmodified. Such modified groups are described, for example, in Eckstein et al., U.S. Pat. No. 5,672,695 and Karpeisky et al., WO 98/28317, respectively, which are both incorporated by reference herein in their entireties.

Various modifications to nucleic acid (e.g., nucleic acid sensor molecule) structure can be made to enhance the utility of these molecules. Such modifications enhance shelf-life, half-life in vitro, stability, and ease of introduction of such oligonucleotides to the target site, e.g., to enhance penetration of cellular membranes, and confer the ability to recognize and bind to targeted cells.

### EXAMPLES

The following examples describe non-limiting examples of methods that are used to isolate nucleic acid aptamers and nucleic acid sensor molecules that are used to detect a drug compound, such as ecstasy, in biological fluides. The aptamers and nucleic acid sensor molecules are designed to discriminate ecstasy or ecstacy metabolites from other over the counter or prescription medications having similar structure, such as ritalin, pseudoepherine, phenylpropanolamine (PPA), propanolol, or nor-pseudoephedrine.

### Example 1

Ecstasy Aptamer Selection

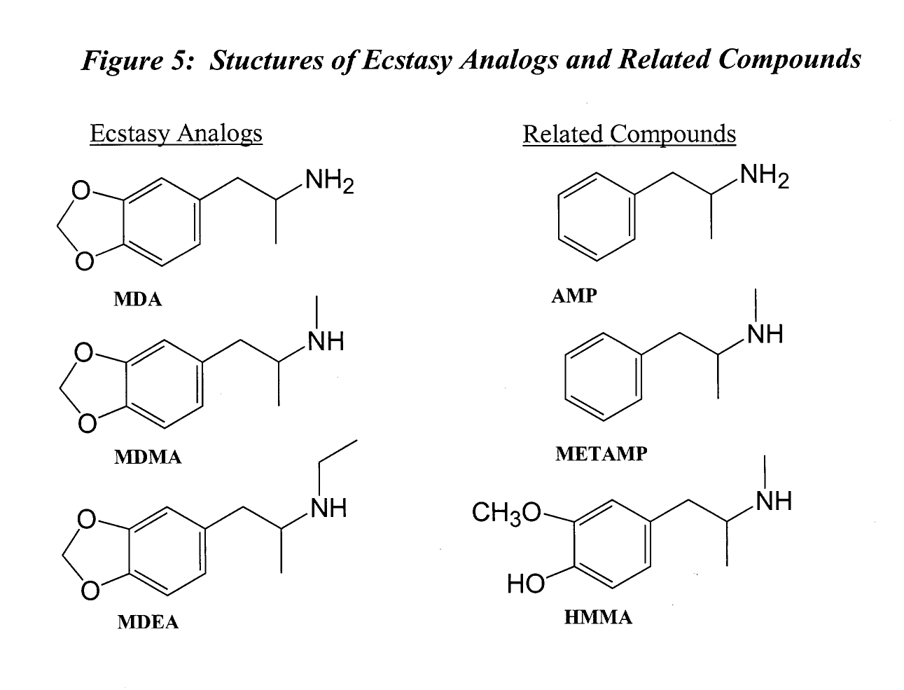

A nucleic acid aptamer that selectively binds MDA, MDMA, MDEA, and/or HMMA (FIG. 5) is provided in accordance with the present invention. The binding affinity of the aptamer for these compounds is preferably represented by the dissociation constant of about 50 nanomolar (nM) or less, and more preferably about 10 nM or less. In one embodiment, the Kd of the aptamer and drug target is established using a double filter nitrocellulose filter binding assay such as that disclosed by Wong and Lohman, 1993, PNAS USA, 90, 5428-5432.

Generally, the method for isolating aptamers of the invention having specificity for ecstasy analogs comprises: (a) preparing a candidate mixture of potential oligonucleotide ligands for ecstasy wherein the candidate mixture is complex enough to contain at least one oligonucleotide ligand for ecstasy or analogs thereof (the target); (b) contacting the candidate mixture with the target under conditions suitable for at least one oligonucleotide in the candidate mixture to bind to the target; (c) removing unbound oligonucleotides from the candidate mixture; (d) collecting the oligonucleotide ligands that are bound to the target to produce a first collected mixture of oligonucleotide ligands; (e) contacting the mixture from (d) with the target under more stringent binding conditions than in (b), wherein oligonucleotide ligands having increased affinity to the target relative to the first collected mixture of (d); (f) removing unbound oligonucleotides from (e); and (g) collecting the oligonucleotide ligands that are bound to the target to produce a second collected mixture of oligonucleotide ligands to thereby identify oligonucleotides having specificity for ecstasy and ecstacy analogs. The method can comprise additional steps in which the oligonucleotides isolated in the first or second collected mixture are enriched or expanded by any suitable technique, such as amplification or mutagenesis, prior to contacting the first collected oligonucleotide mixture with the target under the higher stringency conditions, after collecting the oligonucleotides that bound to the target under the higher stringency conditions, or both. Optionally, the contacting and expanding or enriching steps are repeated as necessary to produce the desired aptamer. Thus, it is possible that the second collected oligonucleotide mixture can comprise a single aptamer. The conditions used to affect the stringency of binding used in the method can include varying reaction conditions used for binding, for example the composition of a buffer, temperature, time, and concentration of the components used for binding can be optimized for the desired level of stringency.

In vitro Selection

In a non-limiting example, aptamers having binding specificity for an ecstasy drug target are isolated by applying the method under the following conditions. First, the ecstasy target is attached to a solid matrix such as a bead or chip surface by means of a covalent (eg. amide or morpholino bond) or non-covalent (eg. biotin/streptavidin) linkage. The structure of MDA, MDMA, and MDEA all share an amino groug that can be used for such coupling, thereby exposing the common methylenedioxyamphetamine face of the molecule. Either a mixture of ecstasy analogs or a single analog or metabolite (HMMA) can be used to generate aptamers of the invention.

A random pool of DNA oligomers is synthesized where the 5′ and 3′ proximal ends are fixed sequences used for amplification and the central region consists of randomized positions. Ten picomoles of template are PCR amplified for 8 cycles in the initial round. Copy DNA of the selected pool of RNA from subsequent rounds of amplification are PCR amplified 18 cycles. PCR reactions are carried out in a 50 .mu.l volume containing 200 picomoles of each primer, 2 mM final concentration dNTP's, 5 units of Thermus aquaticus DNA polymerase (Perkin Elmer Cetus) in a PCR buffer (10 mM Tris-Cl pH 8.4, 50 mM KCl, 7.5 mM MgCl.sub.2, 0.05 mg/ml BSA). Primers are annealed at 58° C. for 20 seconds and extended at 74° C. for 2 minutes. Denaturation can occur at 93° C. for 30 seconds.

Products from PCR amplification are used for T7 in vitro transcription in a 200 ul reaction volume. T7 transcripts are purified from an 8 percent, 7M Urea polyacrylamide gel and eluted by crushing gel pieces in a Sodium Acetate/EDTA solution. For each round of amplification, 50 picomoles of the selected pool of RNA is phosphatased for 30 minutes using Calf Intestinal Alkaline Phosphatase. The reaction is then phenol extracted 3 times and chloroform extracted once, then ethanol precipitated. 25 picomoles of this RNA is 5′ end-labeled using γ-³²P ATP with T4 polynucleotide kinase for 30 minutes. Kinased RNA is gel purified and a small quantity (about 150 fmoles; 100,000 cpm) is used along with 250 picomoles of cold RNA to follow the fraction of RNA bound to the ecstasy target and retained on nitrocellulose filters during the separation step of the method. Typically a target molecule concentration is used that binds one to five percent of the total input RNA. A control (without target) is used to determine the background which is typically 0.1% of the total input. Selected RNA is eluted from the filter by extracting three times with water saturated phenol containing 2% lauryl sulfate (SDS), 0.3M NaOAc and 5 mM EDTA followed by a chloroform extraction. Twenty five percent of this RNA is then used to synthesize cDNA for PCR amplification.

Selection with Non-Amplifiable Competitor RNA

In a non-limiting example, selections are performed using two buffer conditions where the only difference between the buffers is sodium concentration (250 mM NaCl or 500 mM NaCl). Two different buffer conditions are used to increase stringency (with the higher salt concentration being more stringent) and to determine whether different ligands can be obtained. After 10 rounds of amplification, the binding constant of the selected pool can decrease by about an order of magnitude and can remain constant for the next two additional rounds. Competitor RNA is not used in the first 12 rounds. After this round, the pool is split and selection carried out in the presence and absence (control) of competitor RNA. For rounds 12 through 18, a 50-fold excess of a non-amplifiable random pool of RNA is present during selection to compete with non-specific low-affinity binders that may survive and thus be amplified. The competitor RNA, which had a 30N random region, is made as described above for the amplifiable pool RNA; however, the competitor RNA has different primer annealing sequences. Thus, the competitor RNA does not survive the cDNA synthesis or PCR amplification steps. It would be apparent to one skilled in the art that other primer sequences could be used as long as they are not homologous to those used for the pool RNA. The use of competitor RNA can increase the affinity of the selected pool by several orders of magnitude.

Cloning and Sequencing

In a non-limiting example, PCR amplified DNA from the last round selected-pool of RNA is phenol and chloroform extracted and ethanol precipitated. The extracted PCR DNA is then digested using Bam HI and Hind III restriction enzymes and sub-cloned into pUC18. DNAs are phenol and chloroform extracted following digestion. Ligation is carried out at room temperature for two hours after which time the reaction is phenol and chloroform extracted and used to electroporate competent cells. Fifty transformants from the selections using competitor RNA at both NaCl concentrations are picked and their DNAs sequenced.

Binding Assays

In a non-limiting example, binding assays were performed by adding 5 ul of the ecstacy target, at the appropriate concentrations (i.e., ranging from 2×10⁻⁶ with 3 fold dilutions to 9×10⁻⁹ for 250 mM NaCl and 0.5×10⁻⁷ with 3 fold dilutions to 2×10⁻¹⁰ for 50 mM NaCl), to 45 ul of binding buffer (50 mM Na-HEPES pH 7.5, 250 mM NaCl, 2 mM DTT, 10 mM MnCl₂, 5 mM CHAPS) on ice, then adding 50,000 cpm of kinased RNA (<200 fmoles) in a volume of 3 to 4 ul. This mix was incubated at 37° C. for 20 minutes. The reactions were then passed over nitrocellulose filters, which are pre-equilibrated in buffer, and washed with a 50 mM Tris-Cl pH 7.5 solution. Filters were dried and counted.

General Considerations in Aptamer Selection

When a consensus sequence is identified, oligonucleotides that contain that sequence can be made by conventional synthetic or recombinant techniques. These aptamers can also function as target-specific aptamers of this invention. Such an aptamer can conserve the entire nucleotide sequence of an isolated aptamer, or can contain one or more additions, deletions or substitutions in the nucleotide sequence, as long as a consensus sequence is conserved. A mixture of such aptamers can also function as target-specific aptamers, wherein the mixture is a set of aptamers with a portion or portions of their nucleotide sequence being random or varying, and a conserved region that contains the consensus sequence. Additionally, secondary aptamers can be synthesized using one or more of the modified bases, sugars and linkages described herein using conventional techniques and those described herein.

In some embodiments of this invention, aptamers can be sequenced or mutagenized to identify consensus regions or domains that are participating in aptamer binding to target, and/or aptamer structure. This information is used for generating second and subsequent pools of aptamers of partially known or predetermined sequence. Sequencing used alone or in combination with the retention and selection processes of this invention, can be used to generate less diverse oligonucleotide pools from which aptamers can be made. Further selection according to these methods can be carried out to generate aptamers having preferred characteristics for diagnostic or therapeutic applications. That is, domains that facilitate, for example, drug delivery could be engineered into the aptamers selected according to this invention.

Although this invention is directed to making aptamers using screening from pools of non-predetermined sequences of oligonucleotides, it also can be used to make second-generation aptamers from pools of known or partially known sequences of oligonucleotides. A pool is considered diverse even if one or both ends of the oligonucleotides comprising it are not identical from one oligonucleotide pool member to another, or if one or both ends of the oligonucleotides comprising the pool are identical with non-identical intermediate regions from one pool member to another. Toward this objective, knowledge of the structure and organization of the target protein can be useful to distinguish features that are important for biochemical pathway inhibition or biological response generation in the first generation aptamers. Structural features can be considered in generating a second (less random) pool of oligonucleotides for generating second round aptamers:

### Example 2

Nucleic Acid Sensor Design Selection

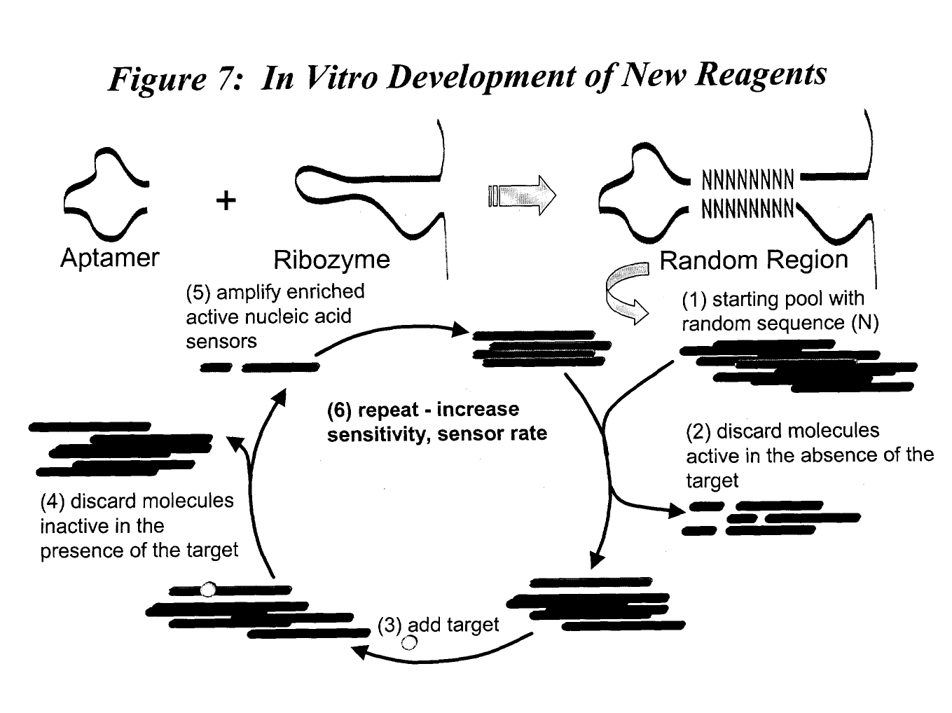

The isolated apatmer obtained from in vitro selection is coupled to the stem-loop II region of a hammerhead ribozyme (FIG. 7) or to a region of another enzymatic nucleic acid motif using a randomized region of nucleotides. The approach is to use an enzymatic nucleic acid molecule where one or more regions are randomized. In this non-limiting example, in vitro selection is applied using a partially randomized RNA population based on the hammerhead self-cleaving ribozyme. The RNA construct used to express the population is designed to take advantage of the fact that the hammerhead ribozyme activity is sensitive to the structure of stem II. In this construct, stem II is replaced with two random-sequence domains that are separated by the aptamer sequence isolated above (sensor domain).

Selection Protocol for Isolation of Ecstasy-Dependent Nucleic Acid Sensor Molecules

Negative Selection: The starting RNA population is comprised of greater than 1012 sequence variants. The RNA population is combined (final concentration=10 μM) with reaction buffer (50 mM Tris-HCl [pH 7.5 at 23° C.]; 20 mM MgCl2) and incubated at 23° C. for 15 hr and the reaction products separated by denaturing (7 M urea) 10% polyacrylamide gel electrophoresis (PAGE). The uncleaved RNA is isolated by excising the precursor (uncleaved RNA) band and the RNA is recovered by a standard crush/soak method. The resulting RNA is precipitated using ethanol and the dried pellet resuspended in deionized water (dH2O).

Positive Selection: The negative-selected RNA population is combined (final concentration=10 μM) with reaction buffer (50 mM Tris-HCl [pH 7.5 at 23° C.]; 20 mM MgCl2). The ecstasy effector molecule is then added (final concentration of 1 μM) to initiate the reaction comprising incubation at 23° C. for 15 min and the reaction products separated by denaturing PAGE. The cleaved RNA is isolated by excising the appropriate cleavage product band and recovering the RNA by a standard crush/soak method. The resulting RNA is precipitated using ethanol and the dried pellet resuspended in deionized water (dH2O).

Amplification: Reverse transcription and polymerase chain reaction (RT-PCR) protocols are conducted according to standard methods. The resulting double-stranded DNA is used as template for in vitro transcription with T7 RNA polymerase under standard reaction conditions.

Protocol Variations: Various parameters of the protocol can be altered to apply selective pressure on specific characteristics of the nucleic acid sensor molecules. For example, decreasing incubation time during positive selection will favor the isolation of nucleic acid sensor molecules with higher rate constants when bound to the effector. Increasing incubation time during negative selection will favor the isolation of nucleic acid sensor molecules that have lower rate constants for nucleic acid sensor molecule cleavage in the absence of effector. Lowering the effector concentration will favor the isolation of nucleic acid sensor molecules with improved affinity for the effector.

In the current example, early rounds of selection use 15 minute incubation for the positive selection reaction. This is progressively reduced to favor the isolation of higher-speed nucleic acid sensor molecules. Also, early rounds of selection make use of a separate reaction buffer wherein Tris is added first, effector is added next, and Mg2+ is added last. This protocol can give rise to a population of nucleic acid sensor molecules that largely requires this order of addition (nucleic acid sensor molecules do not become active if Tris and Mg2+ are added in combination, followed by addition of effector). In later rounds, the order of addition is altered as outlined above, and this change permitted the selection of nucleic acid sensor molecules that are able to switch from the OFF state to the ON state when effector is applied.

An essential component of the selection process is the use of modified negative selection protocols that disfavor the isolation of selfish molecules. For example, in later rounds, negative selection reactions are employed that comprise repetitive cycles (3 to 5) of ˜1 hr incubation at 23° C. followed by a 30 sec incubation at 90° C. This is expected to permit misfolded RNAs to become denatured and refolded in order to maximize the removal of nucleic acid sensor molecules that do not require effector to cleave.

### Example 4

Selection of Ecstasy Dependent Nucleic Acid Sensor Molecules

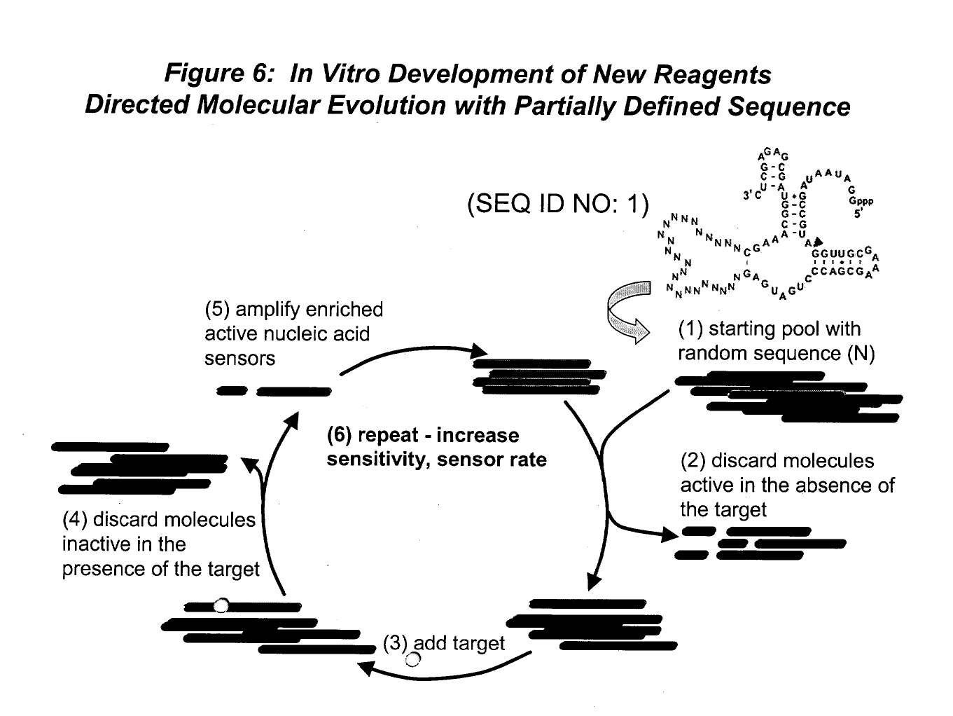

A nucleic acid sensor molecule is generated by in vitro selection techniques to be active only in the presence of ecstasy or an ecstasy metabolite. In this non-limiting example, in vitro selection is applied using a partially randomized RNA population based on the hammerhead self-cleaving ribozyme (FIG. 6). The RNA construct used to express the population is designed to take advantage of the fact that the hammerhead ribozyme activity is sensitive to the structure of stem II. In this construct, stem II is replaced with one or more randomized sensor domain.

Selection Protocol for Isolation of Ecstasy-Dependent Nucleic Acid Sensor Molecules

Negative Selection: The starting RNA population is comprised of greater than 1012 sequence variants. The RNA population is combined (final concentration=10 μM) with reaction buffer (50 mM Tris-HCl [pH 7.5 at 23° C.]; 20 mM MgCl2) and incubated at 23° C. for 15 hr and the reaction products separated by denaturing (7 M urea) 10% polyacrylamide gel electrophoresis (PAGE). The uncleaved RNA is isolated by excising the precursor (uncleaved RNA) band and the RNA is recovered by a standard crush/soak method. The resulting RNA is precipitated using ethanol and the dried pellet resuspended in deionized water (dH2O).

Positive Selection: The negative-selected RNA population is combined (final concentration=10 μM) with reaction buffer (50 mM Tris-HCl [pH 7.5 at 23° C.]; 20 mM MgCl2). The ecstasy effector molecule is then added (final concentration of 1 μM) to initiate the reaction comprising incubation at 23° C. for 15 min and the reaction products separated by denaturing PAGE. The cleaved RNA is isolated by excising the appropriate cleavage product band and recovering the RNA by a standard crush/soak method. The resulting RNA is precipitated using ethanol and the dried pellet resuspended in deionized water (dH2O).

Amplification: Reverse transcription and polymerase chain reaction (RT-PCR) protocols are conducted according to standard methods. The resulting double-stranded DNA is used as template for in vitro transcription with T7 RNA polymerase under standard reaction conditions.

Protocol Variations: Various parameters of the protocol can be altered to apply selective pressure on specific characteristics of the nucleic acid sensor molecules. For example, decreasing incubation time during positive selection will favor the isolation of nucleic acid sensor molecules with higher rate constants when bound to the effector. Increasing incubation time during negative selection will favor the isolation of nucleic acid sensor molecules that have lower rate constants for nucleic acid sensor molecule cleavage in the absence of effector. Lowering the effector concentration will favor the isolation of nucleic acid sensor molecules with improved affinity for the effector.

In the current example, early rounds of selection use 15 minute incubation for the positive selection reaction. This is progressively reduced to favor the isolation of higher-speed nucleic acid sensor molecules. Also, early rounds of selection make use of a separate reaction buffer wherein Tris is added first, effector is added next, and Mg2+ is added last. This protocol can give rise to a population of nucleic acid sensor molecules that largely requires this order of addition (nucleic acid sensor molecules do not become active if Tris and Mg2+ are added in combination, followed by addition of effector). In later rounds, the order of addition is altered as outlined above, and this change permitted the selection of nucleic acid sensor molecules that are able to switch from the OFF state to the ON state when effector is applied.

An essential component of the selection process is the use of modified negative selection protocols that disfavor the isolation of selfish molecules. For example, in later rounds, negative selection reactions are employed that comprise repetitive cycles (3 to 5) of ˜1 hr incubation at 23° C. followed by a 30 sec incubation at 90° C. This is expected to permit misfolded RNAs to become denatured and refolded in order to maximize the removal of nucleic acid sensor molecules that do not require effector to cleave.

### Example 5

Selection of Cocaine Dependent Nucleic Acid Sensor Molecules

A nucleic acid sensor molecule is generated by in vitro selection techniques to be active only in the presence of cocaine or a cocaine metabolite. In this non-limiting example, in vitro selection is applied using a partially randomized RNA population based on the hammerhead self-cleaving ribozyme (FIG. 6). The RNA construct used to express the population is designed to take advantage of the fact that the hammerhead ribozyme activity is sensitive to the structure of stem II. In this construct, stem II is replaced with one or more randomized sensor domain.

Selection Protocol for Isolation of Cocaine-Dependent Nucleic Acid Sensor Molecules

Negative Selection: The starting RNA population is comprised of greater than 1012 sequence variants. The RNA population is combined (final concentration=10 μM) with reaction buffer (50 mM Tris-HCl [pH 7.5 at 23° C.]; 20 mM MgCl2) and incubated at 23° C. for 15 hr and the reaction products separated by denaturing (7 M urea) 10% polyacrylamide gel electrophoresis (PAGE). The uncleaved RNA is isolated by excising the precursor (uncleaved RNA) band and the RNA is recovered by a standard crush/soak method. The resulting RNA is precipitated using ethanol and the dried pellet resuspended in deionized water (dH2O).

Positive Selection: The negative-selected RNA population is combined (final concentration=10 μM) with reaction buffer (50 mM Tris-HCl [pH 7.5 at 23° C.]; 20 mM MgCl2). The cocaine effector molecule is then added (final concentration of 1 μM) to initiate the reaction comprising incubation at 23° C. for 15 min and the reaction products separated by denaturing PAGE. The cleaved RNA is isolated by excising the appropriate cleavage product band and recovering the RNA by a standard crush/soak method. The resulting RNA is precipitated using ethanol and the dried pellet resuspended in deionized water (dH2O).

Amplification: Reverse transcription and polymerase chain reaction (RT-PCR) protocols are conducted according to standard methods. The resulting double-stranded DNA is used as template for in vitro transcription with T7 RNA polymerase under standard reaction conditions.

Protocol Variations: Various parameters of the protocol can be altered to apply selective pressure on specific characteristics of the nucleic acid sensor molecules. For example, decreasing incubation time during positive selection will favor the isolation of nucleic acid sensor molecules with higher rate constants when bound to the effector. Increasing incubation time during negative selection will favor the isolation of nucleic acid sensor molecules that have lower rate constants for nucleic acid sensor molecule cleavage in the absence of effector. Lowering the effector concentration will favor the isolation of nucleic acid sensor molecules with improved affinity for the effector.

In the current example, early rounds of selection use 15 minute incubation for the positive selection reaction. This is progressively reduced to favor the isolation of higher-speed nucleic acid sensor molecules. Also, early rounds of selection make use of a separate reaction buffer wherein Tris is added first, effector is added next, and Mg2+ is added last. This protocol can give rise to a population of nucleic acid sensor molecules that largely requires this order of addition (nucleic acid sensor molecules do not become active if Tris and Mg2+ are added in combination, followed by addition of effector). In later rounds, the order of addition is altered as outlined above, and this change permitted the selection of nucleic acid sensor molecules that are able to switch from the OFF state to the ON state when effector is applied.

An essential component of the selection process is the use of modified negative selection protocols that disfavor the isolation of selfish molecules. For example, in later rounds, negative selection reactions are employed that comprise repetitive cycles (3 to 5) of ˜1 hr incubation at 23° C. followed by a 30 sec incubation at 90° C. This is expected to permit misfolded RNAs to become denatured and refolded in order to maximize the removal of nucleic acid sensor molecules that do not require effector to cleave.

### Example 6

Oxycontin Aptamer Selection

A nucleic acid aptamer that selectively binds Oxycontin and/or a Oxycontin metabolite is provided in accordance with the present invention. The binding affinity of the aptamer for these compounds is preferably represented by the dissociation constant of about 50 nanomolar (nM) or less, and more preferably about 10 nM or less. In one embodiment, the Kd of the aptamer and drug target is established using a double filter nitrocellulose filter binding assay such as that disclosed by Wong and Lohman, 1993, PNAS USA, 90, 5428-5432.

Generally, the method for isolating aptamers of the invention having specificity for Oxycontin comprises: (a) preparing a candidate mixture of potential oligonucleotide ligands for Oxycontin wherein the candidate mixture is complex enough to contain at least one oligonucleotide ligand for Oxycontin or analogs/metabolites thereof (the target); (b) contacting the candidate mixture with the target under conditions suitable for at least one oligonucleotide in the candidate mixture to bind to the target; (c) removing unbound oligonucleotides from the candidate mixture; (d) collecting the oligonucleotide ligands that are bound to the target to produce a first collected mixture of oligonucleotide ligands; (e) contacting the mixture from (d) with the target under more stringent binding conditions than in (b), wherein oligonucleotide ligands having increased affinity to the target relative to the first collected mixture of (d); (f) removing unbound oligonucleotides from (e); and (g) collecting the oligonucleotide ligands that are bound to the target to produce a second collected mixture of oligonucleotide ligands to thereby identify oligonucleotides having specificity for Oxycontin and Oxycontin analogs/metabolites. The method can comprise additional steps in which the oligonucleotides isolated in the first or second collected mixture are enriched or expanded by any suitable technique, such as amplification or mutagenesis, prior to contacting the first collected oligonucleotide mixture with the target under the higher stringency conditions, after collecting the oligonucleotides that bound to the target under the higher stringency conditions, or both. Optionally, the contacting and expanding or enriching steps are repeated as necessary to produce the desired aptamer. Thus, it is possible that the second collected oligonucleotide mixture can comprise a single aptamer. The conditions used to affect the stringency of binding used in the method can include varying reaction conditions used for binding, for example the composition of a buffer, temperature, time, and concentration of the components used for binding can be optimized for the desired level of stringency.

In vitro Selection

In a non-limiting example, aptamers having binding specificity for an Oxycontin drug target are isolated by applying the method under the following conditions. First, the Oxycontin target is attached to a solid matrix such as a bead or chip surface by means of a covalent (eg. amide or morpholino bond) or non-covalent (eg. biotin/streptavidin) linkage. Either a mixture of Oxycontin analogs or a single analog or metabolite can be used to generate aptamers of the invention.

A random pool of DNA oligomers is synthesized where the 5′ and 3′ proximal ends are fixed sequences used for amplification and the central region consists of randomized positions. Ten picomoles of template are PCR amplified for 8 cycles in the initial round. Copy DNA of the selected pool of RNA from subsequent rounds of amplification are PCR amplified 18 cycles. PCR reactions are carried out in a 50 .mu.l volume containing 200 picomoles of each primer, 2 mM final concentration dNTP's, 5 units of Therrnus aquaticus DNA polymerase (Perkin Elmer Cetus) in a PCR buffer (10 mM Tris-Cl pH 8.4, 50 mM KCl, 7.5 mM MgCl.sub.2, 0.05 mg/ml BSA). Primers are annealed at 58.degree. C. for 20 seconds and extended at 74.degree. C. for 2 minutes. Denaturation can occur at 93.degrees. C. for 30 seconds.

Products from PCR amplification are used for T7 in vitro transcription in a 200 ul reaction volume. T7 transcripts are purified from an 8 percent, 7M Urea polyacrylamide gel and eluted by crushing gel pieces in a Sodium Acetate/EDTA solution. For each round of amplification, 50 picomoles of the selected pool of RNA is phosphatased for 30 minutes using Calf Intestinal Alkaline Phosphatase. The reaction is then phenol extracted 3 times and chloroform extracted once, then ethanol precipitated. 25 picomoles of this RNA is 5′ end-labeled using .gamma ³²P ATP with T4 polynucleotide kinase for 30 minutes. Kinased RNA is gel purified and a small quantity (about 150 finoles; 100,000 cpm) is used along with 250 picomoles of cold RNA to follow the fraction of RNA bound to the Oxycontin target and retained on nitrocellulose filters during the separation step of the method. Typically a target molecule concentration is used that binds one to five percent of the total input RNA. A control (without target) is used to determine the background which is typically 0.1% of the total input. Selected RNA is eluted from the filter by extracting three times with water saturated phenol containing 2% lauryl sulfate (SDS), 0.3M NaOAc and 5 mM EDTA followed by a chloroform extraction. Twenty five percent of this RNA is then used to synthesize cDNA for PCR amplification.

Selection with Non-Amplifiable Competitor RNA

In a non-limiting example, selections are performed using two buffer conditions where the only difference between the buffers is sodium concentration (250 mM NaCl or 500 mM NaCl). Two different buffer conditions are used to increase stringency (with the higher salt concentration being more stringent) and to determine whether different ligands can be obtained. After 10 rounds of amplification, the binding constant of the selected pool can decrease by about an order of magnitude and can remain constant for the next two additional rounds. Competitor RNA is not used in the first 12 rounds. After this round, the pool is split and selection carried out in the presence and absence (control) of competitor RNA. For rounds 12 through 18, a 50-fold excess of a non-amplifiable random pool of RNA is present during selection to compete with non-specific low-affinity binders that may survive and thus be amplified. The competitor RNA, which had a 30N random region, is made as described above for the amplifiable pool RNA; however, the competitor RNA has different primer annealing sequences. Thus, the competitor RNA does not survive the cDNA synthesis or PCR amplification steps. It would be apparent to one skilled in the art that other primer sequences could be used as long as they are not homologous to those used for the pool RNA. The use of competitor RNA can increase the affinity of the selected pool by several orders of magnitude.

Cloning and Sequencing

In a non-limiting example, PCR amplified DNA from the last round selected-pool of RNA is phenol and chloroform extracted and ethanol precipitated. The extracted PCR DNA is then digested using Bam HI and Hind III restriction enzymes and sub-cloned into pUC18. DNAs are phenol and chloroform extracted following digestion. Ligation is carried out at room temperature for two hours after which time the reaction is phenol and chloroform extracted and used to electroporate competent cells. Fifty transformants from the selections using competitor RNA at both NaCl concentrations are picked and their DNAs sequenced.

Binding Assays

In a non-limiting example, binding assays are performed by adding 5 ul of the Oxycontin target, at the appropriate concentrations (i.e., ranging from 2×10⁻⁶ with 3 fold dilutions to 9×10⁻⁹ for 250 mM NaCl and 0.5×10⁻⁷ with 3 fold dilutions to 2×10⁻¹⁰ for 50 mM NaCl), to 45 ul of binding buffer (50 mM Na-HEPES pH 7.5, 250 mM NaCl, 2 mM DTT, 10 mM MnCl₂, 5 mM CHAPS) on ice, then adding 50,000 cpm of kinased RNA (<200 fmoles) in a volume of 3 to 4 ul. This minx is incubated at 37.degrees C. for 20 minutes. The reactions are then passed over nitrocellulose filters, which are pre-equilibrated in buffer, and washed with a 50 mM Tris-Cl pH 7.5 solution. Filters are dried and counted.

General Considerations in Aptamer Selection

When a consensus sequence is identified, oligonucleotides that contain that sequence can be made by conventional synthetic or recombinant techniques. These aptamers can also function as target-specific aptamers of this invention. Such an aptamer can conserve the entire nucleotide sequence of an isolated aptamer, or can contain one or more additions, deletions or substitutions in the nucleotide sequence, as long as a consensus sequence is conserved. A mixture of such aptamers can also function as target-specific aptamers, wherein the mixture is a set of aptamers with a portion or portions of their nucleotide sequence being random or varying, and a conserved region that contains the consensus sequence. Additionally, secondary aptamers can be synthesized using one or more of the modified bases, sugars and linkages described herein using conventional techniques and those described herein.

In some embodiments of this invention, aptamers can be sequenced or mutagenized to identify consensus regions or domains that are participating in aptamer binding to target, and/or aptamer structure. This information is used for generating second and subsequent pools of aptamers of partially known or predetermined sequence. Sequencing used alone or in combination with the retention and selection processes of this invention, can be used to generate less diverse oligonucleotide pools from which aptamers can be made. Further selection according to these methods can be carried out to generate aptamers having preferred characteristics for diagnostic or therapeutic applications. That is, domains that facilitate, for example, drug delivery could be engineered into the aptamers selected according to this invention.

Although this invention is directed to making aptamers using screening from pools of non-predetermined sequences of oligonucleotides, it also can be used to make second-generation aptamers from pools of known or partially known sequences of oligonucleotides. A pool is considered diverse even if one or both ends of the oligonucleotides comprising it are not identical from one oligonucleotide pool member to another, or if one or both ends of the oligonucleotides comprising the pool are identical with non-identical intermediate regions from one pool member to another. Toward this objective, knowledge of the structure and organization of the target protein can be useful to distinguish features that are important for biochemical pathway inhibition or biological response generation in the first generation aptamers. Structural features can be considered in generating a second (less random) pool of oligonucleotides for generating second round aptamers:

### Example 7

Selection of Oxycontin Dependent Nucleic Acid Sensor Molecules

A nucleic acid sensor molecule is generated by in vitro selection techniques to be active only in the presence of oxycontin or an oxycontin metabolite. As described above, a apatmer with specificity for Oxycontin can be used to generate a nucleic acid sensor molecule as shown in FIG. 7. As an alternative, in this non-limiting example, in vitro selection is applied using a partially randomized RNA population based on the hammerhead self-cleaving ribozyme (FIG. 6). The RNA construct used to express the population is designed to take advantage of the fact that the hammerhead ribozyme activity is sensitive to the structure of stem II. In this construct, stem II is replaced with one or more randomized sensor domain.

Selection Protocol for Isolation of Oxycontin-Dependent Nucleic Acid Sensor Molecules

Negative Selection: The starting RNA population is comprised of greater than 1012 sequence variants. The RNA population is combined (final concentration=10 μM) with reaction buffer (50 mM Tris-HCl [pH 7.5 at 23° C.]; 20 mM MgCl2) and incubated at 23° C. for 15 hr and the reaction products separated by denaturing (7 M urea) 10% polyacrylamide gel electrophoresis (PAGE). The uncleaved RNA is isolated by excising the precursor (uncleaved RNA) band and the RNA is recovered by a standard crush/soak method. The resulting RNA is precipitated using ethanol and the dried pellet resuspended in deionized water (dH2O).

Positive Selection: The negative-selected RNA population is combined (final concentration=10 μM) with reaction buffer (50 mM Tris-HCl [pH 7.5 at 23° C.]; 20 mM MgCl2). The oxycontin effector molecule is then added (final concentration of 1 μM) to initiate the reaction comprising incubation at 23° C. for 15 min and the reaction products separated by denaturing PAGE. The cleaved RNA is isolated by excising the appropriate cleavage product band and recovering the RNA by a standard crush/soak method. The resulting RNA is precipitated using ethanol and the dried pellet resuspended in deionized water (dH2O).

Amplification: Reverse transcription and polymerase chain reaction (RT-PCR) protocols are conducted according to standard methods. The resulting double-stranded DNA is used as template for in vitro transcription with T7 RNA polymerase under standard reaction conditions.

Protocol Variations: Various parameters of the protocol can be altered to apply selective pressure on specific characteristics of the nucleic acid sensor molecules. For example, decreasing incubation time during positive selection will favor the isolation of nucleic acid sensor molecules with higher rate constants when bound to the effector. Increasing incubation time during negative selection will favor the isolation of nucleic acid sensor molecules that have lower rate constants for nucleic acid sensor molecule cleavage in the absence of effector. Lowering the effector concentration will favor the isolation of nucleic acid sensor molecules with improved affinity for the effector.

In the current example, early rounds of selection use 15 minute incubation for the positive selection reaction. This is progressively reduced to favor the isolation of higher-speed nucleic acid sensor molecules. Also, early rounds of selection make use of a separate reaction buffer wherein Tris is added first, effector is added next, and Mg2+ is added last. This protocol can give rise to a population of nucleic acid sensor molecules that largely requires this order of addition (nucleic acid sensor molecules do not become active if Tris and Mg2+ are added in combination, followed by addition of effector). In later rounds, the order of addition is altered as outlined above, and this change permitted the selection of nucleic acid sensor molecules that are able to switch from the OFF state to the ON state when effector is applied.

An essential component of the selection process is the use of modified negative selection protocols that disfavor the isolation of selfish molecules. For example, in later rounds, negative selection reactions are employed that comprise repetitive cycles (3 to 5) of ˜1 hr incubation at 23° C. followed by a 30 sec incubation at 90° C. This is expected to permit misfolded RNAs to become denatured and refolded in order to maximize the removal of nucleic acid sensor molecules that do not require effector to cleave.

### Example 8

Detection of Ecstasy in Biologial Fluids

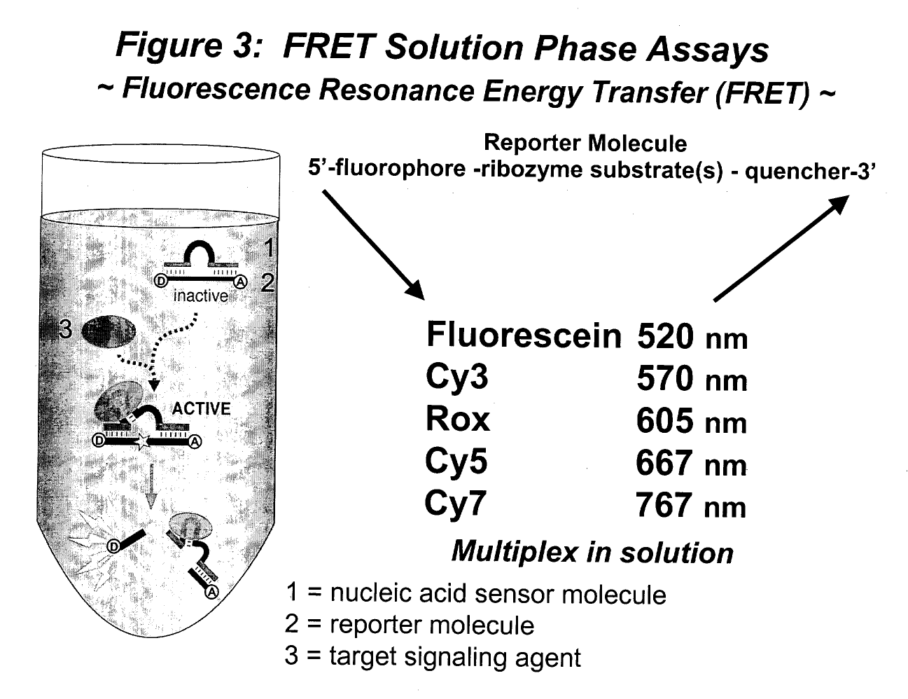

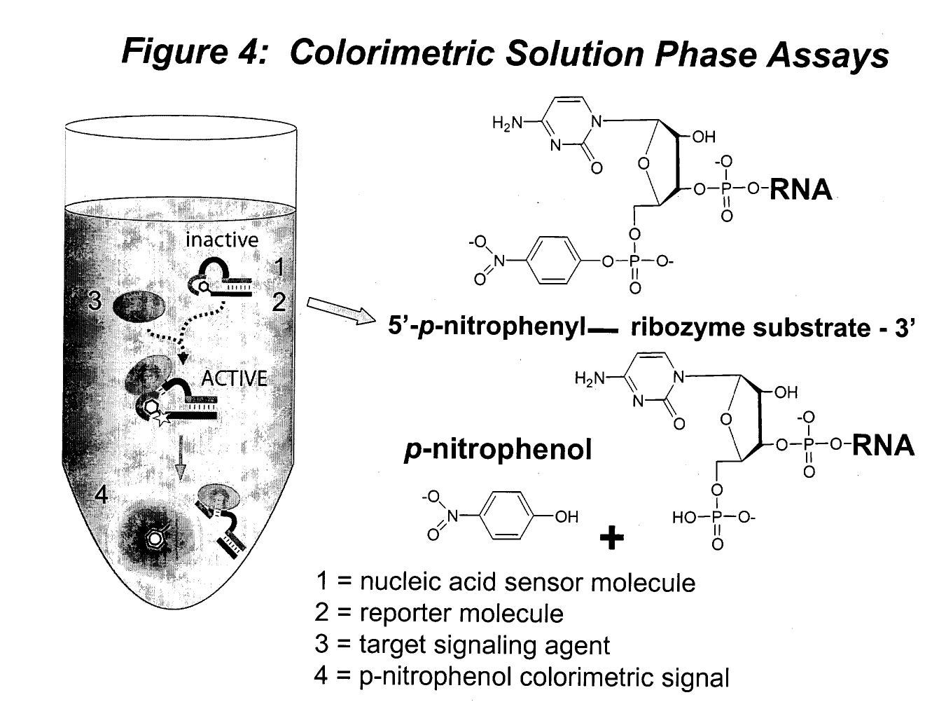

Nucleic acid sensor molecules are used to assay the presence of ecstasy (MDA, MDMA, and/or MDEA) in biological fluids. The nucleic acid sensor molecule is designed to detect the presence of ecstasy using florescence resonance energy transfer (FRET) as shown in FIG. 3. Alternately, a colorimetric detection scheme is used as shown in FIG. 4. The assay is designed such that either all ecstasy analogs can be detected, for example MDA, MDMA and MDEA, or the metabolite HMMA can be detected, as distinguished from other compounds such as amphetamine, methamphetamine, ephedrine, psuedoephedrine, etc. (FIG. 5).

In a non-limiting example, a saliva or urine sample is collected from a subject. This sample is then used as a component of a kit comprising a nucleic acid sensor molecule and reporter molecule of the invention, along with any other reagents such as buffers and excipients that are suited for the assay. The sample can be diluted with a buffer or used neat, and can optionally be partially purified or neutralized as the assay may require. The sample is then contacted with the nucleic acid sensor molecule under conditions suitable for detection of the target drug. In the presence of the target molecule, the nucleic acid sensor molecule catalyses a reaction that is detected by standard techniques, for example cleaving a nucleic acid substate comprising FRET moieties (FIG. 3) or by colorimetric assay (FIG. 4). The amount of signal is quantitated using a standard curve generated using known quantities of the effector molecule, for example an amount between 100 and 5,000 ug/l.

The assay kit can comprise various devices, compartments, wells, channels or vessels to contain the various components of the kit and combine components when necessary. For example, the kit can comprise a device that allows loading of the sample followed by contact with the nucleic acid sensor and analytic read-out. Such a device can operate via liquid/liquid phase interaction or solid phase/liquid phase interaction using adsorption media or interactions on a surface. Devices for colorimetric and/or UV assay are also contemplated by the methods of the invention, as are automated processes of detection known in the art.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The methods and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. Thus, such additional embodiments are within the scope of the present invention and the following claims.

The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

TABLE I
Exemplary Drug Compounds (substances of abuse)

| | |
|---|---|
| 4-MTA (4-methylthioamphetamine) | Methadone |
| Alpha-ethyltryptamine | Methamphetamine |
| Amphetamine | Methaqualone |
| Amyl nitrite | Methcathinone |
| Benzocaine | Methylphenidate (ritalin) |
| Cocaine | Morphine |
| Dimethyltryptamine | Nexus (2CB) |
| Ectasy (MDA, MDMA, MDEA) | Nicotine |
| Ephedrine | Opium |
| Erythropoietine (Epogen) | Oxycodone |
| Fentanyl | OxyContin |
| Gamma Hydroxybutyrate (GHB) | PCP (phencyclidine) |
| GBL (Gamma butyrolactone) | Peyote |
| GHB (Gamma Hydroxybutyrate) | Phenobarbital |
| Hashish | Procaine |
| Heroin | Psilocybin |
| Isobutyl nitrite | Psilocybin/psilocin |
| Ketamine | Pseudoephedrine |
| Lidocaine | Ritalin |
| LSD (Lysergic acid diethylamide) | Rohypnol |
| Mannitol | Scopolamine |
| Marijuana (THC) | Steroids |
| Mescaline | Strychnine |
| | Talwin |

TABLE II
A. 2.5 μmol Synthesis Cycle ABI 394 Instrument

| Reagent | Equivalents | Amount | Wait Time* DNA | Wait Time* 2′-O-methyl | Wait Time*RNA |
|---|---|---|---|---|---|
| Phosphoramidites | 6.5 | 163 μL | 45 sec | 2.5 min | 7.5 min |
| S-Ethyl Tetrazole | 23.8 | 238 μL | 45 sec | 2.5 min | 7.5 min |
| Acetic Anhydride | 100 | 233 μL | 5 sec | 5 sec | 5 sec |
| N-Methyl | 186 | 233 μL | 5 sec | 5 sec | 5 sec |
| imidazole | | | | | |
| TCA | 176 | 2.3 mL | 21 sec | 21 sec | 21 sec |
| Iodine | 11.2 | 1.7 mL | 45 sec | 45 sec | 45 sec |
| Beaucage | 12.9 | 645 μL | 100 sec | 300 sec | 300 sec |
| Acetonitrile | NA | 6.67 mL | NA | NA | NA |

B. 0.2 μmol Synthesis Cycle ABI 394 Instrument

| Reagent | Equivalents | Amount | Wait Time* DNS | Wait Time* 2′-O-methyl | Wait Time*RNA |
|---|---|---|---|---|---|
| Phosporamidites | 15 | 31 μL | 45 sec | 233 sec | 465 sec |
| S-Ethyl Tetrazole | 38.7 | 31 μL | 45 sec | 233 min | 465 sec |
| Acetic Anhydride | 655 | 124 μL | 5 sec | 5 sec | 5 sec |
| N-Methyl | 1245 | 124 μL | 5 sec | 5 sec | 5 sec |
| Imidazole | | | | | |
| TCA | 700 | 732 μL | 10 sec | 10 sec | 10 sec |
| Iodine | 20.6 | 244 μL | 15 sec | 15 sec | 15 sec |
| Beaucage | 7.7 | 232 μL | 100 sec | 300 sec | 300 sec |
| Acetonitrile | NA | 2.64 mL | NA | NA | NA |

C. 0.2 μmol Synthesis Cycle 96 well Instrument

| | Equivalents:DNA/ | Amount: DNA/2′-O- | | Wait Time* 2′-O- | Wait Time* |
|---|---|---|---|---|---|
| Reagent | 2′-O-methyl/Ribo | methy/Ribo | Wait Time* DNA | methyl | Ribo |
| Phosphoramidites | 22/33/66 | 40/60/120 μL | 60 sec | 180 sec | 360 sec |
| S-Ethyl Tetrazole | 70/105/210 | 40/60/120 μL | 60 sec | 180 min | 360 sec |
| Acetic Anhydride | 265/265/265 | 50/50/50 μL | 10 sec | 10 sec | 10 sec |
| N-Methyl | 502/502/502 | 50/50/50 μL | 10 sec | 10 sec | 10 sec |
| Imidazole | | | | | |
| TCA | 238/475/475 | 250/500/500 μL | 15 sec | 15 sec | 15 sec |
| Iodine | 6.8/6.8/6.8 | 80/80/80 μL | 30 sec | 30 sec | 30 sec |
| Beaucage | 34/51/51 | 80/120/120 | 100 sec | 200 sec | 200 sec |
| Acetonitrile | NA | 1150/1150/1150 μL | NA | NA | NA |

·Wait time does not include contact time during delivery.

I claim:

1. A method of detecting the presence of a compound in a biological sample, the method comprising: (a) contacting the sample with an nucleic acid sensor molecule, and (b) assaying for the presence of the drug under conditions suitable for detecting the presence of the drug in the sample, wherein the nucleic acid sensor molecule comprises a enzymatic nucleic acid component and one or more sensor components that upon interaction with the compound induces a chemical reaction of the enzymatic nucleic acid component that modulates the activity or properties of the reporter molecule, signaling the presence of the compound.

2. The method according to claim 1 wherein the sensor component or components have a greater interaction affinity for the compound as compared to other compounds in the biological sample.

3. The method according to claim 1 wherein the compound is a drug.

4. The method according to claim 3 wherein the drug is ecstasy.


SEQUENCE LISTING (1 sequence)

SEQ ID NO: 1 - RNA, 83 nt, Artificial Sequence
  Description of Artificial Sequence  Enzymatic Nucleic Acid
ggauaauagccguagguugcgaaagcgacccugaugagnnnnnnnnnnnnnnnnnnnnnnnnncgaaacgguagcgagagcuc